SEQUENCE LISTING (27 sequences)

SEQ ID NO: 1 - protein, 127 aa, DROSOPHILA
PVIVFINPKSGGNQGHKLLGKFQHLLNPRQVFDLTQGGPKMGLDMFRKAPNLRVLACGGDGTVGWVLSVLDQIQPPLQPAPAVGVLPLGTGNDLARALGWGGGYTDEPIGKILREIGMSQCVLMDRW

SEQ ID NO: 2 - protein, 125 aa, mammalian
PLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRW

SEQ ID NO: 3 - protein, 132 aa, mammalian
PLIILANSRSGTNMGEGLLGEFRILLNPVQVFDVTKTPPIKALQLCTLLPYYSARVLVCGGDGTVGWVLDAVDDMKIKGQEKYIPQVAVLPLGTGNDLSNTLGWGTGYAGEIPVAQVLRNVMEADGIKLDRW

SEQ ID NO: 4 - protein, 138 aa, mammalian
RVLVLLNPRGGKGKALQLFRSHVQPLLAEAEISFTLMLTERRNHARELVRSEELGRWDALVVMSGDGLMHEVVNGLMERPDWETAIQKPLCSLPAGSGNALAASLNHYAGYEQVTNEDLLTNCTLLLCRRLLSPMNLL

SEQ ID NO: 5 - protein, 138 aa, mammalian
RVLVLLNPQGGKGKALQLFQSRVQPFLEEAEITFKLILTERKNHARELVCAEELGHWDALAVMSGDGLMHEVVNGLMERPDWETAIQKPLCSLPGGSGNALAASVNHYAGYEQVTNEDLLINCTLLLCRRRLSPMNLL

SEQ ID NO: 6 - protein, 138 aa, mammalian
RLLLLVNPFGGRGLAWQWCKNHVLPMISEAGLSFNLIQTERQNHARELVQGLSLSEWDGIVTVSGDGLLHEVLNGLLDRPDWEEAVKMPVGILPCGSGNALAGAVNQHGGFEPALGLDLLLNCSLLLCRGGGHPLDLL

SEQ ID NO: 7 - protein, 138 aa, mammalian
RLLILVNPFGGRGLAWQRCMDHVVPMISEAGLSFNLIQTERQNHARELVQGLSLSEWEGIVTVSGDGLLYEVLNGLLDRPDWEDAVRMPIGVLPCGSGNALAGAVSHHGGFEQVVGVDLLLNCSLLLCRGGSHPLDLL

SEQ ID NO: 8 - protein, 133 aa, yeast
SILVIINPHGGKGTAKNLFLTKARPILVESGCKIEIAYTKYARHAIDIAKDLDISKYDTIACASGDGIPYEVINGLYRRPDRVDAFNKLAVTQLPCGSGNAMSISCHWTNNPSYAALCLVKSIETRIDLMCCS

SEQ ID NO: 9 - protein, 133 aa, yeast
SIFVIINPFGGKGKAKKLFMTKAKPLLLASRCSIEVVYTKYPGHAIEIAREMDIDKYDTIACASGDGIPHEVINGLYQRPDHVKAFNNIAITEIPCGSGNAMSVSCHWTNNPSYSTLCLIKSIETRIDLMCCS

SEQ ID NO: 10 - protein, 132 aa, S. pombe
RFIVFINPHGGKGKAKHIWESEAEPVFSSAHSICEVVLTRRKDHAKSIAKNLDVGSYDGILSVGGDGLFHEVINGLGERDDYLEAFKLPVCMIPGGSGNAFSYNATGQLKPALTALEILKGRPTSFDLMTFE

SEQ ID NO: 11 - protein, 138 aa, C. elegans
NLLVFINPNSGTGKSLETFANTVGPKLDKSLIRYEVVVTTGPNHARNVLMTKADLGKFNGVLILSGDGLVFEALNGILCREDAFRIFPTLPIGIVPSGSGNGLLCSVLSKYGTKMNEKSVMERALEIATSPTAKAESV

SEQ ID NO: 12 - protein, 137 aa, Arabidosis
RLLVFVNPFGGKKSAREIFVKEVKPLFEDADVQLEIQETKYQLHAKEFVKSMDVSKYDGIVCVSGDGILVEVVNGLLERADWRNALKLPIGMVPAGTGNGMIKSLLDTVGLRCCANSATISIIRGHKRSVDVATIAQ

SEQ ID NO: 13 - DNA, 24 bp, mammalian
gaacccgcggggcgccaagggcaa

SEQ ID NO: 14 - DNA, 27 bp, mammalian
gctgaacccccggggcgacaagggcaa

SEQ ID NO: 15 - DNA, 21 bp, mammalian
cgcggcggcgccggcaaggcc

SEQ ID NO: 16 - DNA, 21 bp, mammalian
ggcaagggcgccgccttgcag

SEQ ID NO: 17 - DNA, 24 bp, mammalian
gtggtcatggccggcgacgggctg

SEQ ID NO: 18 - DNA, 30 bp, mammalian
gtggtcatggatggagacggcctgatgcac

SEQ ID NO: 19 - DNA, 19 bp, mammalian
tcatgtctgcagacgggct

SEQ ID NO: 20 - DNA, 26 bp, mammalian
tcatgtctgacgacggcctgatgcac

SEQ ID NO: 21 - DNA, 23 bp, mammalian
gtctggagatgcattgatgcacg

SEQ ID NO: 22 - DNA, 20 bp, mammalian
ctggagacgatctgatgcac

SEQ ID NO: 23 - DNA, 21 bp, mammalian
gccatccaggcccccctgtgt

SEQ ID NO: 24 - DNA, 24 bp, mammalian
gccatccagcggccgctgtgtagc

SEQ ID NO: 25 - DNA, 23 bp, mammalian
agcctccctgcagcctctggcaa

SEQ ID NO: 26 - DNA, 19 bp, mammalian
tcccagcagactctggcaa

SEQ ID NO: 27 - DNA, 24 bp, mammalian
cccagcaggatccgacaactcgct

(19) United States
(12) Patent Application Publication
(10) Pub. No.: US 2008/0279841 A1
(43) Pub. Date: Nov. 13, 2008

(54) Novel Therapeutic Molecular Variants And Uses Thereof

(75) Inventors: Stuart Pitson, South Australia (AU); Paul Moretti, South Australia (AU); Julia Zebol, South Australia (AU); Pu Xia, South Australia (AU); Jennifer Gamble, South Australia (AU); Mathew Vadas, South Australia (AU); Richard D'Andrea, South Australia (AU); Binks Wattenberg, South Australia (AU)

Correspondence Address: MORRISON & FOERSTER LLP, 1650 TYSONS BOULEVARD, SUITE 400, MCLEAN, VA, 22102, US

(73) Assignee: MEDVET SCIENCE PTY LTD., Thebarton, South Australia (AU)

(21) Appl. No.: 10/312,336

(22) PCT Filed: Jun. 20, 2001
(86) PCT No.: PCT/AU01/00730

§ 371 Date: Aug. 19, 2003

(30) Foreign Application Priority Data
Jun. 28, 2000 (AU) PQ 8408
Jul. 11, 2000 (AU) PQ 8699
Sep. 8, 2000 (AU) PQ 9980
Jan. 29, 2001 (AU) PR 2749

(51) Int. Cl. A61K 38/45; C12N 9/12; C12N 15/54; C12Q 1/48; G01N 33/53

(52) U.S. Cl. 424/94.5; 435/194; 536/23.2; 435/15; 435/7.1

(57) ABSTRACT

The present invention relates generally to a sphingosine kinase variant and to derivatives, analogues, chemical equivalents and mimetics thereof exhibiting reduced catalytic activity and, more particularly, to sphingosine kinase variants which exhibit a reduced capacity to phosphorylate sphingosine to sphingosine-1-phosphate. The present invention also contemplates genetic sequences encoding said sphingosine kinase variants and derivatives, analogues and mimetics thereof. The variants of the present invention are useful in a range of therapeutic and prophylactic applications.

### FIELD OF THE INVENTION

The present invention relates generally to a sphingosine kinase variant and to derivatives, analogues, chemical equivalents and mimetics thereof exhibiting reduced catalytic activity and, more particularly, to sphingosine kinase variants which exhibit a reduced capacity to phosphorylate sphingosine to sphingosine-1-phosphate. The present invention also contemplates genetic sequences encoding said sphingosine kinase variants and derivatives, analogues and mimetics thereof. The variants of the present invention are useful in a range of therapeutic and prophylactic applications.

### BACKGROUND OF THE INVENTION

Bibliographic details of the publications referred to by author in this specification are collected at the end of the description.

The reference to any prior art in this specification is not, and should not be taken as, an acknowledgment or any form of suggestion that prior art forms part of the common general knowledge in Australia.

Sphingosine kinase is a key regulatory enzyme in a variety of cellular responses. Sphingosine-1-phosphate is known to be an important second messenger in signal transduction (Meyer et al., 1997). It is mitogenic in various cell types (Alessenko, 1998) and appears to trigger a diverse range of important regulatory pathways including prevention of ceramide-induced apoptosis (Culliver et al., 1996), mobilisation of intracellular calcium by an IP₃-independant pathway, stimulation of DNA synthesis, activation of mitogen-activated protein (MAP) kinase pathway, activation of phospholipase D, and regulation of cell motility (for reviews see (Meyer et al., 1997; Spiegal et al., 1998; Igarashi, 1997)).

Recent studies (Xia et al., 1998) have shown that sphingosine-1-phosphate is an obligatory signalling intermediate in the inflammatory response of vascular endothelial cells to tumour necrosis factor-α (TNFα). In spite of its obvious importance, very little is known of the mechanisms that control cellular sphingosine-1-phosphate levels. It is known that sphingosine-1-phosphate levels in the cell are mediated largely by its formation from sphingosine by sphingosine kinase, and to a lesser extent by its degradation by endoplasmic reticulum-associated sphingosine-1-phosphate lyase and sphingosine-1-phosphate phosphatase (Spiegel et al., 1998). Basal levels of sphingosine-1-phosphate in the cell are generally low, but can increase rapidly and transiently when cells are exposed to mitogenic agents. This response appears correlated with an increase in sphingosine kinase activity in the cytosol and can be prevented by addition of the sphingosine kinase inhibitory molecules N,N-dimethylsphingosine and DL-threo-dihydrosphingosine. This indicates that sphingosine kinase is an important molecule responsible for regulating cellular sphingosine-1-phosphate levels. This places sphingosine kinase in a central and obligatory role in mediating the effects attributed to sphingosine-1-phosphate in the cell.

Sphigosine kinase is speculated to play a role in a number of cellular activities including inflammation, calcium mobilisation, cell motility and adhesion molecule expression. Accordingly, there is a need to develop mechanisms of regulating these cellular activities via regulation of the sphingosine kinase signalling pathway.

In work leading up to the present invention, the inventors have determined that amino acid sequence mutations introduced into the amino acid region defined by amino acid 16-153 of the human sphingosine kinase protein result in the production of a sphingosine kinase variant which, in addition to exhibiting no sphingosine kinase baseline functional activity, also suppresses activation of wild-type sphingosine kinase molecules. Accordingly, the sphingosine kinase variants of the present invention both provide novel molecules for use in modulating sphingosine kinase signalling pathway function and facilitate the screening for and/or rational analysis, design and/or modification of agents for use in either effectively mutating wild-type sphingosine kinase molecules or mimicking the activity of sphingosine kinase variant molecules.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Specific mutations in amino acid sequence are represented herein as “Xaa₁nXaa₂” where Xaa₁ is the original amino acid residue before mutation, n is the residue number and Xaa₂ is the mutant amino acid. The abbreviation “Xaa” may be the three letter or single letter amino acid code. A mutation in single letter code is represented, for example, by X₁nX₂ where X₁ and X₂ are the same as Xaa₁ and Xaa₂, respectively. The amino acid residues for sphingosine kinase are numbered with the residue glycine in the motif Asp Gly Leu Met (DGLM) being residue number 82.

The subject specification contains nucleotide and amino acid sequence information prepared using the programme Patent In Version 2.0, presented herein after the bibliography. Each nucleotide or amino acid sequence is identified in the sequence listing by the numeric indicator <210> followed by the sequence identifier (e.g. <210>1, <210>2, etc). The length, type of sequence (DNA, protein (PRT), etc) and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400>followed by the sequence identifier (e.g. <400>1, <400>2, etc).

One aspect of the present invention is directed to a sphingosine kinase variant comprising a mutation in a region defined by amino acids 16-153 or functionally equivalent region wherein said variant exhibits ablated or reduced catalytic activity relative to wild-type sphingosine kinase or a derivative, homologue, analogue, chemical equivalent or mimetic of said sphingosine kinase variant.

Another aspect of the present invention provides a human sphingosine kinase variant comprising a mutation in a region defined by amino acids 16-153 or functionally equivalent region wherein said variant exhibits ablated or reduced catalytic activity relative to wild-type human sphingosine kinase or a derivative, homologue, analogue, chemical equivalent or mimetic of said sphingosine kinase variant.

In a preferred embodiment there is provided a human sphingosine kinase variant comprising an amino acid sequence with a single or multiple amino. acid substitution, addition and/or deletion in a region defined by amino acids 16-153 or functionally equivalent region wherein said variant exhibits ablated or reduced catalytic activity relative to wild-type sphingosine kinase or a derivative, homologue, analogue, chemical equivalent or mimetic of said sphingosine kinase variant.

In still a more preferred embodiment, there is provided a human sphingosine kinase variant comprising an amino acid sequence of the single or multiple amino acid substitution, addition and/or deletion in a region defined by amino acids 70-90, and more preferably 79-84, or functionally equivalent region wherein said variant exhibits ablated or reduced catalytic activity relative to wild-type sphingosine kinase or a derivative, homologue, analogue, chemical equivalent or mimetic of said sphingosine kinase variant.

In another preferred embodiment there is provided a human sphingosine kinase variant comprising an amino acid sequence with a single or multiple amino acid substitution, addition and/or deletion in a region defined by amino acid 16-153 or functionally equivalent region wherein said variant exhibits ablated catalytic activity relative to wild-type sphingosine kinase or a derivative, homologue, analogue, chemical equivalent or mimetic of said sphingosine kinase variant.

In a most preferred embodiment, the subject sphingosine kinase variant comprises one or more of the amino acid substitutions selected from the following list:

### (i) G82D

### (ii) G82A

(iii) G26D

### (iv) S79D

### (v) G80D

### (vi) K103A

(vii) G111D
(viii) G113D

### (ix) G26A

### (x) K27A

### (xi) K29A

(xii) S79A
(xiii) G80A
(xiv) K103R

### (xv) G111A

In another aspect the present invention is directed to a sphingosine kinase variant comprising a mutation in an ATP binding site region or functionally equivalent region wherein said variant exhibits ablated or reduced catalytic activity relative to wild-type sphingosine kinase or a derivative, homologue, analogue, chemical equivalent or mimetic of said sphingosine kinase variant.

Another aspect of the present invention is directed to an isolated nucleic acid molecule selected from the list consisting of:
(i) An isolated nucleic acid molecule or derivative or equivalent thereof comprising a nucleotide sequence encoding or complementary to a sequence encoding a sphingosine kinase variant or derivative, homologue, analogue, chemical equivalent or mimetic of said variant which variant comprises a mutation in a region defined by amino acid 16-153 or functionally equivalent region wherein said variant exhibits ablated or reduced catalytic activity relative to wild-type sphingosine kinase.
(ii) An isolated nucleic acid molecule or derivative or equivalent thereof comprising a nucleotide sequence encoding or complementary to a sequence encoding a human sphingosine kinase variant or derivative, homologue, analogue, chemical equivalent or mimetic of said variant which variant comprises a mutation in a region defined by amino acid 16-153 or functionally equivalent region wherein said variant exhibits ablated or reduced catalytic activity relative to wild-type human sphingosine kinase.
(iii) An isolated nucleic acid molecule or derivative or equivalent thereof comprising a nucleotide sequence encoding or complementary to a sequence encoding a human sphingosine kinase variant or derivative, homologue, analogue, chemical equivalent or mimetic of said variant, which variant comprises an amino acid sequence with a single or multiple amino acid substitution, addition and/or deletion in a region defined by amino acid 16-153 or functionally equivalent region wherein said variant exhibits ablated or reduced catalytic activity relative to wild-type sphingosine kinase.
(iv) An isolated nucleic acid molecule or derivative or equivalent thereof comprising a nucleotide sequence encoding or complementary to a sequence encoding a human sphingosine kinase variant or derivative, homologue, analogue, chemical equivalent or mimetic of said variant, which variant comprises an amino acid sequence with a single or multiple amino acid substitution, addition and/or deletion in a region defined by amino acid 70-90 or functionally equivalent region wherein said variant exhibits ablated or reduced catalytic activity relative to wild-type sphingosine kinase.
(v) An isolated nucleic acid molecule or derivative or equivalent thereof comprising a nucleotide sequence encoding or complementary to a sequence encoding a human sphingosine kinase variant or derivative, homologue, analogue, chemical equivalent or mimetic of said variant, which variant comprises an amino acid sequence with a single or multiple amino acid substitution, addition and/or deletion in a region defined by amino acid 79-84 or functionally equivalent region wherein said variant exhibits ablated or reduced catalytic activity relative to wild-type sphingosine kinase.
(vi) An isolated nucleic acid molecule or derivative or equivalent thereof comprising a nucleotide sequence encoding or complementary to a sequence encoding a sphingosine kinase variant or a derivative, homologue, analogue, chemical equivalent or mimetic of said variant comprising one or more of the amino acid substitutions selected from the following list:
   (a) G82D
   (b) G82A
   (c) G26D
   (d) S79D
   (e) G80D
   (f) K103A
   (g) G111D
   (h) G113D
   (i) G26A
   (j) K27A
   (k) K29A
   (l) S79A
   (m) G80A
   (n) K103R
   (o) G111A
(vii) An isolated nucleic acid molecule or derivative or analogue thereof comprising a nucleotide sequence encoding or complementary to a sequence encoding a sphingosine kinase variant or derivative, homologue, analogue, chemical equivalent or mimetic of said variant which variant comprises a mutation in an ATP binding site region or functionally equivalent region wherein said variant exhibits ablated or reduced catalytic activity relative to wild-type sphingosine kinase.

Accordingly, another aspect of the present invention provides a method for detecting an agent capable of modulating the interaction of FOSK with sphingosine kinase or its functional equivalent or derivative thereof said method comprising contacting a cell or extract thereof containing said sphingosine kinase and FOSK or its functional equivalent or derivative with a putative agent and detecting an altered expression phenotype associated with said interaction.

In yet another aspect the present invention provides a method for detecting an agent capable of binding or otherwise associating with the sphingosine kinase region defined by amino acids 16-153 or functional equivalent or derivative thereof said method comprising contacting a cell containing said amino acid region or functional equivalent or derivative thereof with a putative agent and detecting an altered expression phenotype associated with modulation of the function of sphingosine kinase or its functional equivalent or derivative.

Accordingly, another aspect of the present invention is directed to a method for analysing, designing and/or modifying an agent capable of interacting with the sphingosine kinase region defined by amino acids 16-153 or derivative thereof and modulating at least one functional activity associated with said sphingosine kinase said method comprising contacting said sphingosine kinase or derivative thereof with a putative agent and assessing the degree of interactive complementarity of said agent with said binding site.

In a related aspect, the present invention should be understood to extend to the agents identified utilising any of the methods hereinbefore defined. In this regard, reference to an agent should be understood as a reference to any proteinaceous or non-proteinaceous molecule which modulates at least one sphingosine kinase functional activity.

Another aspect of the present invention contemplates a method of modulating cellular functional activity in a mammal said method comprising administering to said mammal an effective amount of a sphingosine kinase variant or agent as hereinbefore defined for a time and under conditions sufficient to inhibit, reduce or otherwise down-regulate at least one functional activity of wild-type sphingosine kinase.

Another aspect of the present invention relates to the treatment and/or prophylaxis of a condition in a mammal, which condition is characterised by aberrant, unwanted or otherwise inappropriate cellular activity, said method comprising administering to said mammal an effective amount of a sphingosine kinase variant or agent as hereinbefore defined for a time and under conditions sufficient to inhibit, reduce or otherwise down-regulate at least one functional activity of wild-type sphingosine kinase wherein said down-regulation results in modulation of cellular functional activity.

A further aspect of the present invention relates to the use of a sphingosine kinase variant or agent as hereinbefore defined in the manufacture of a medicament for the modulation of cellular functional activity.

Another aspect of the present invention relates to a sphingosine kinase variant or agent as hereinbefore defined for use in modulating cellular functional activity.

In yet another further aspect the present invention contemplates a pharmaceutical composition comprising a sphingosine kinase variant or agent as hereinbefore defined together with one or more pharmaceutically acceptable carriers and/or diluents.

Single and three letter abbreviations used throughout the specification are defined in Table 1.

TABLE 1
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### BRIEF DESCRIPTION OF THE DRAWINGS

FIG. 1 is a schematic representation of the sequence alignment of the putative catalytic domains of some diacylglycerol kinases with sphingosine kinases. Highly conserved residues within the putative catalytic domain of diacylglycerol kinases are highlighted. The marked () residue indicates the site where mutagenesis (Gly→Asp) in these three diacylglycerol kinases ablates catalytically activity.

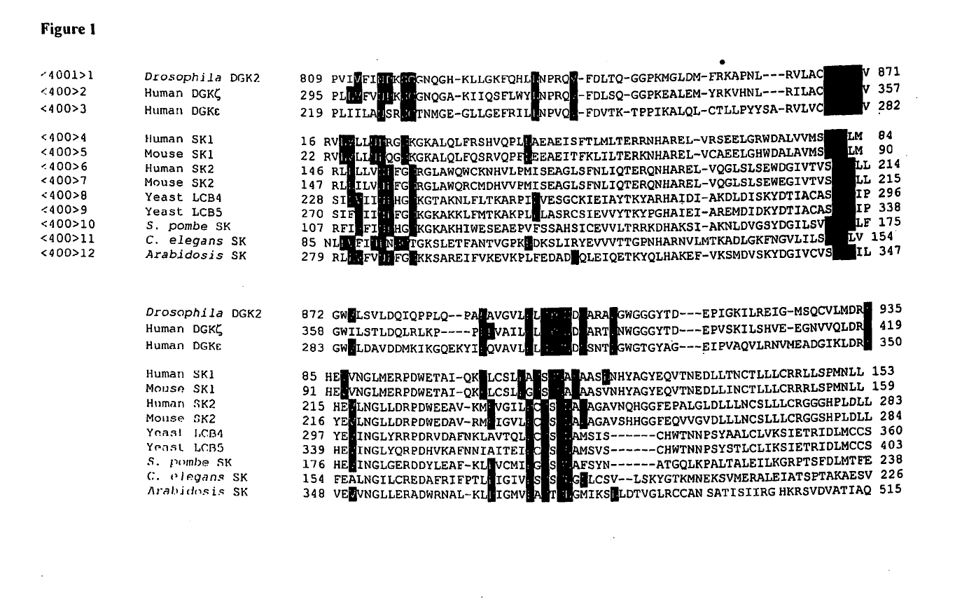

FIG. 2 is both a graphical representation and image of site directed mutagenesis of human sphingosine kinase HEK293 cells transfected with either pcDNA3-SK, pcDNA3-G26DSK, pcDNA3-S79DSK, pcDNA3-G80DSK, pcDNA3-G82DSK, pcDNA3-K103ASK, pcDNA3-G111DSK, pcDNA3-G113DSK, or empty pcDNA3 vector were harvested and analysed for (A) protein expression levels by Western blot using the M2 anti-FLAG antibody, and (B) sphingosine kinase activity.

FIG. 3 is a graphical representation demonstrating that expression of G82D SK in HEK293 cells blocks activation of endogenous sphingosine kinase activity by TNFα, PMA and IL-1. HEK293 cells transfected with either pcDNA3-G82DSK or empty pcDNA3 vector were treated with 1 ng/ml TNFα and 100 units/ml IL-1 for 10 min and 100 ng/ml PMA for 30 min.

FIG. 4 is a graphical representation of time course of sphingosine kinase activation by TNFα in HEK293 cells expressing G82D SK. HEK293 cells transfected with either pcDNA3-G82DSK or empty pcDNA3 vector were treated with 1 ng/ml TNFα. Cells were harvested at various times over 45 min. of TNFα treatment with the cell lysates assayed for sphingosine kinase activity.

FIG. 5 is a graphical representation demonstrating that expression of G82D SK in HEK293 cells decreases activation of overexpressed wild-type sphingosine kinase activity by TNFα. HEK293 cells, either transfected with pcDNA3-SK, or cotransfected with equval proportions of pcDNA3-SK and pcDNA3-G82DSK were treated with 1 ng/ml TNFα for 10 min. Cells were then harvested and the SK activity in the cell lysates determined.

FIG. 6 is a graphical representation that the expression of G82D SK in 3T3 fibroblasts inhibiting activation of SK by the oncogene Ras.

FIG. 7 is a graphical representation that the expression of G82D SK in HEK293 cells does not effect activation of protein kinase C activity by TNFα or PMA.

FIG. 8 is an image demonstrating that the expression of G82D SK in HEK293 cells does not inhibit activation of sphingomyelinase by TNF.

FIG. 9 is a graphical representation demonstrating that the expression of G82D SK in HEK293 prevents ERK activation by TNFα.

FIG. 10 is a schematic representation of the types of drugs which can be identified and/or developed in light of the development of the present invention.

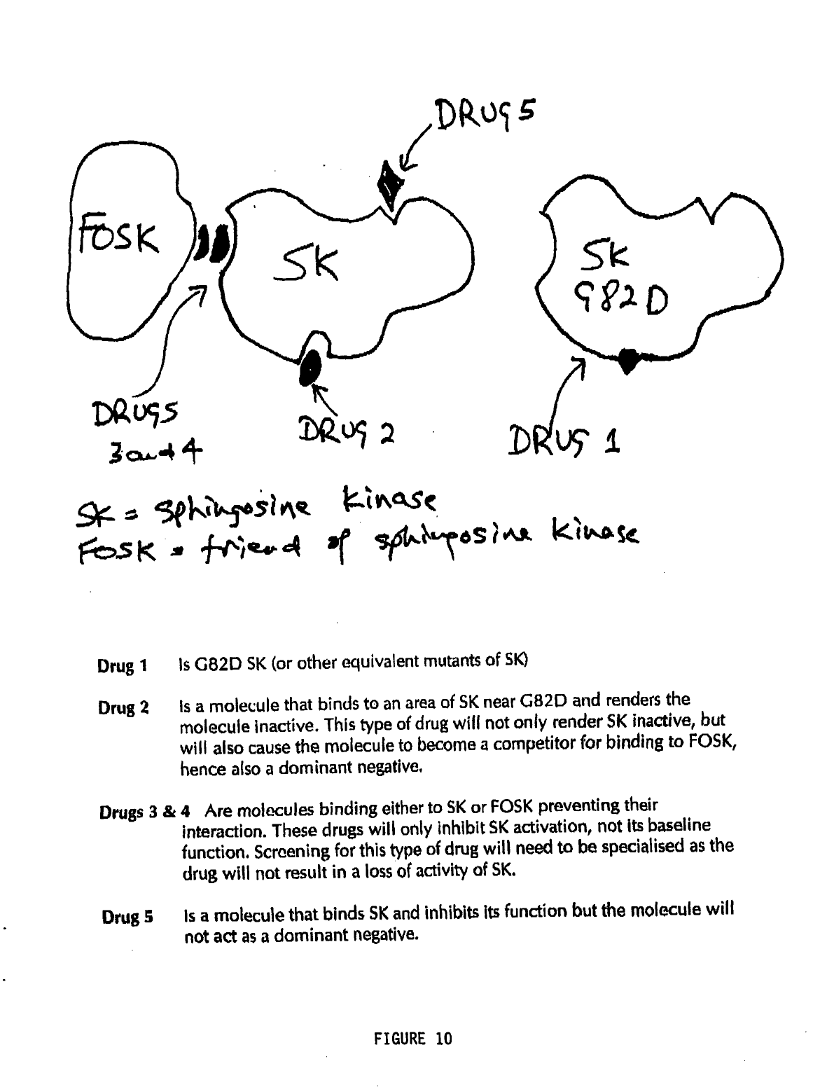

FIG. 11 is a graphical representation demonstrating that G82D SK inhibits Ras transformation. A. NIH 3T3 cells were transfected with V12-Ras, v-Src or V12-Ras plus G82D-SK, SphK activity was measured 48 h after transfection. B. Focus formation assays were performed in V12-Ras, v-Src, SphK, or V12-Ras plus G82D-SK transfected NIH 3T3 cells in the absence or presence of DMS (2.5 μM) over two weeks.

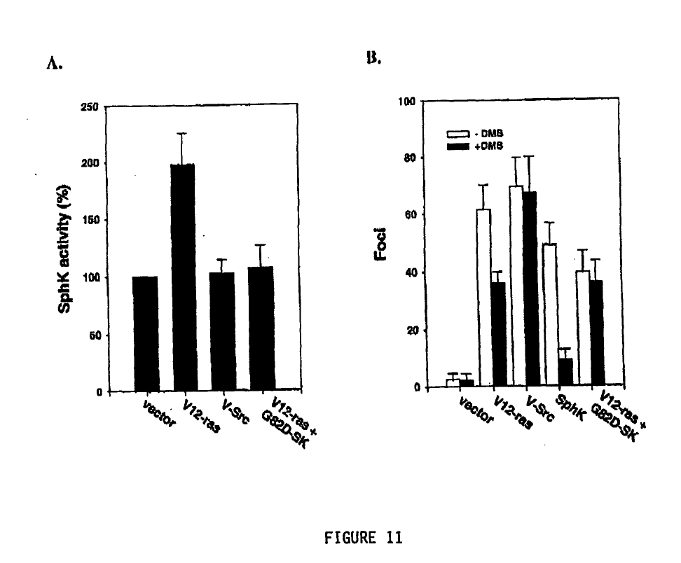

FIG. 12 shows site directed mutagenesis of human sphingosine kinase HEK293T cells transfected with either pcDNA3-SK^{WT}, pcDNA3-SK^{G82D}, pcDNA3-SK^{G82A}, or empty pcDNA3 vector were harvested and analysed for (A) protein expression levels by Western blot using the MT anti-FLAG antibody, and (B) sphingosine kinase activity.

FIG. 13 shows kinetic analysis with ATP of (A) hSK^{WT} and (B) hSK^{G82}A. Kinetic analyses were performed with ATP in the concentration range of 0-2 mM and 0-40 mM for hSK^{WT} and hSK^{G82A}, respectively. In both cases sphingosine was present at 100 μM.

FIG. 14 shows kinetic analysis with sphingosine of (A) hSK^{WT} and (B) hSK^{G82A}. Kinetic analyses were performed with sphingosine in the concentration range of 0-2 mM for both hSK^{WT} and hSK^{G82A}. ATP was present at 1 mM and 40 mM for hSK^{WT} and hSK^{G82A}, respectively.

FIG. 15 is a graphical representation of site directed mutagenesis of human sphingosine kinase. HEK293T cells transfected with either empty pcDNA3 vector, pcDNA3-SK^{WT}, or pcDNA3-mutant hSK were harvested and analysed for sphingosine kinase activity.

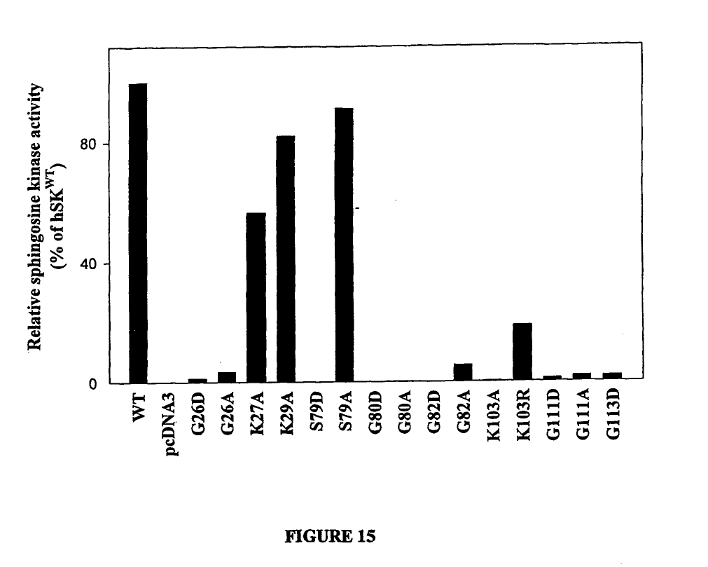

### DETAILED DESCRIPTION OF THE PREFERRED EMBODIMENTS

The present invention is predicated, in part, on the determination that the ablation of catalytic activity of human sphingosine kinase can be achieved by introducing a mutation into the amino acid region defined by amino acids 16-153. Further, the introduction of such a mutation not only generates a sphingosine kinase variant which exhibits ablated, reduced or a limited baseline functional activity but also generates a variant which can function as a dominant negative sphingosine kinase, either in vitro or in vivo, in that it inhibits the activation of wild-type sphingosine kinase. This determination facilitates the rational design of products and methodology for use in the therapy and prophylaxis of conditions characterised by the aberrant, unwanted or otherwise inappropriate functioning of sphingosine kinase signalling.

Accordingly, one aspect of the present invention is directed to a sphingosine kinase variant comprising a mutation in a region defined by amino acids 16-153 or functionally equivalent region wherein said variant exhibits ablated or reduced catalytic activity relative to wild-type sphingosine kinase or a derivative, homologue, analogue, chemical equivalent or mimetic of said sphingosine kinase variant.

Reference to “sphingosine kinase” should be understood as including a reference to all forms of sphingosine kinase protein or derivatives, homologues, analogues, equivalents or mimetics thereof. In this regard, “sphingosine kinase” should be understood as being a molecule which is, inter alia, involved in the generation of sphingosine-1-phosphate during activation of the sphingosine kinase signalling pathway. This includes, for example, all protein forms of sphingosine kinase or its functional derivatives, homologues, analogues, equivalents or mimetics thereof including, for example, any isoforms which arise from alternative splicing of sphingosine kinase mRNA or allelic or polymorphic variants of sphingosine kinase. Preferably, said sphingosine kinase is human sphingosine kinase.

Accordingly there is more particularly provided a human sphingosine kinase variant comprising a mutation in a region defined by amino acids 16-153 or functionally equivalent region wherein said variant exhibits ablated or reduced catalytic activity relative to wild-type human sphingosine kinase or a derivative, homologue, analogue, chemical equivalent or mimetic of said sphingosine kinase variant.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

Reference to “mutation” should be understood as a reference to any change, alteration or other modification, whether occurring naturally or non-naturally, which renders a sphingosine kinase molecule catalytically inactive or capable only of a reduced level of catalytic activity. In this regard, the phrase “catalytic activity” in the context of sphingosine kinase activity should be understood as a reference to the capacity of sphingosine kinase to phosphorylate sphingosine to sphingosine-1 phosphate.

The change, alteration or other modification may take any form including, but not limited to, a structural modification (such an alteration in the secondary, tertiary or quaternary structure of the sphingosine kinase molecule), a molecular modification (such as an addition, substitution or deletion of one or more amino acids from the sphingosine kinase protein) or a chemical modification. The subject modification should also be understood to extend to the fusion, linking or binding of a proteinaceous or non-proteinaceous molecule to the sphingosine kinase protein or to the nucleic acid molecule encoding a sphingosine kinase protein thereby rendering the expression product either catalytically inactive or capable only of reduced catalytic activity. It should also be understood that although it is necessary that the subject mutation is expressed by the sphingosine kinase expression product, the creation of the mutation may be achieved by any suitable means including either mutating a wild-type sphingosine kinase protein, synthesising a sphingosine kinase variant or modifying a nucleic acid molecule encoding a wild-type sphingosine kinase protein such that the expression product of said mutated nucleic acid molecule is a sphingosine kinase protein variant. Preferably, said mutation is a single or multiple amino acid sequence substitution, addition and/or deletion.

In accordance with this preferred embodiment there is provided a human sphingosine kinase variant comprising an amino acid sequence with a single or multiple amino acid substitution, addition and/or deletion in a region defined by amino acids 16-153 or functionally equivalent region wherein said variant exhibits ablated or reduced catalytic activity relative to wild-type sphingosine kinase or a derivative, homologue, analogue, chemical equivalent or mimetic of said sphingosine kinase variant.

In terms of the present invention, reference to “wild-type” sphingosine kinase is a reference to the forms of sphingosine kinase expressed by most individuals in a given population wherein the subject sphingosine kinase is catalytically active within the context discussed hereinbefore. There may be greater than one wild-type form of sphingosine kinase (for example due to allelic or isoform variation) and the level of catalytic activity exhibited by said wild-type sphingosine kinase molecules may fall within a range of levels. However, it should be understood that “wild-type” does not include reference to a naturally occurring form of sphingosine kinase which is not catalytically active. Such a variant form of sphingosine kinase may, in fact, constitute a naturally occurring mutant form of sphingosine kinase within the context of the present invention.

In still a more preferred embodiment, there is provided a human sphingosine kinase variant comprising an amino acid sequence of the single or multiple amino acid substitution, addition and/or deletion in a region defined by amino acids 70-90, and more preferably 79-84, or functionally equivalent region wherein said variant exhibits ablated or reduced catalytic activity relative to wild-type sphingosine kinase or a derivative, homologue, analogue, chemical equivalent or mimetic of said sphingosine kinase variant.

In a most preferred embodiment, the subject sphingosine kinase variant comprises an amino acid substitution of the glycine amino acid at position 82 to aspartic acid.

Without limiting the invention to any one theory or mode of action, sphingosine kinase is thought to exhibit two levels of catalytic activity. At the first level, sphingosine kinase exhibits baseline catalytic activity. At the second level, sphingosine kinase exhibiting baseline activity can be activated such that the Vmax of the enzyme is increased. In the context of the present invention, the ablation or reduction of sphingosine kinase catalytic activity will be achieved where the baseline activity and/or the activation of sphingosine kinase beyond that of baseline activity is ablated or reduced. Preferably, both levels of activity are ablated or reduced and even more preferably both levels of activity are ablated.

In another preferred embodiment there is provided a human sphingosine kinase variant comprising an amino acid sequence with a single or multiple amino acid substitution, addition and/or deletion in a region defined by amino acid 16-153 or functionally equivalent region wherein said variant exhibits ablated catalytic activity relative to wild-type sphingosine kinase or a derivative, homologue, analogue, chemical equivalent or mimetic of said sphingosine kinase variant.

Preferably said subject human sphingosine kinase variant comprises an amino acid addition, substitution and/or deletion in the region defined by amino acids 70-90 and even more preferably 79-84.

In a most preferred embodiment, the subject sphingosine kinase variant comprises one or more of the amino acid substitutions selected from the following list:

### (i) G82D

### (ii) G82A

(iii) G26D

### (iv) S79D

### (v) G80D

### (vi) K103A

(vii) G111D
(viii) G113D

### (ix) G26A

### (x) K27A

### (xi) K29A

(xii) S79A
(xiii) G80A
(xiv) K103R

### (xv) G111A

“Derivatives” include fragments, parts, portions, variants and mimetics from natural, synthetic or recombinant sources including fusion proteins. Parts or fragments include, for example, active regions of sphingosine kinase. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Reference to “homologues” should be understood as a reference to sphingosine kinase nucleic acid molecules or proteins derived from species other than the species being treated.

Chemical and functional equivalents of sphingosine kinase nucleic acid or protein molecules should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl- -aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

Still without limiting the present invention to any one theory or mode of action, it is thought that the human wild-type sphingosine kinase protein region defined by amino acid residues 16-153 comprises all or part of an ATP binding site. Accordingly, it is thought that by blocking the ATP binding site, the subject sphingosine kinase is rendered catalytically inactive in terms of its capacity to phosphorylate sphingosine to sphingosine-1-phosphate. The phrase “functionally equivalent region” should therefore be understood as a reference to any region of a sphingosine kinase amino acid sequence which exhibits at least one of the function activities attributable to the region defined by amino acid residue numbers 16-153.

Accordingly, in another. aspect the present invention is directed to a sphingosine kinase variant comprising a mutation in an ATP binding site region or functionally equivalent region wherein said variant exhibits ablated or reduced catalytic activity relative to wild-type sphingosine kinase or a derivative, homologue, analogue, chemical equivalent or mimetic of said sphingosine kinase variant.

Preferably, said sphingosine kinase is a human sphingosine kinase.

Still more preferably, said mutation is a substitution, deletion and/or addition of one or more amino acids in the region defined by amino acid residues 16-153, more preferably 70-90 and still more preferably 79-84.

In a most preferred embodiment, said mutation comprises one or more of the amino acid substitutions selected from the following list:

### (i) G82D

### (ii) G82A

(iii) G26D

### (iv) S79D

### (v) G80D

### (vi) K103A

(vii) G111D
(viii) G113D

### (ix) G26A

### (x) K27A

### (xi) K29A

(xii) S79A
(xiii) G80A
(xiv) K103R

### (xv) G111A

To the extent that the present invention relates to sphingosine kinase variants comprising one or more amino acid additions, substitutions and/or deletions, it should also be understood to extend to nucleic acid molecules encoding said variants.

Accordingly, another aspect of the present invention is directed to an isolated nucleic acid molecule selected from the list consisting of:
(i) An isolated nucleic acid molecule or derivative or equivalent thereof comprising a nucleotide sequence encoding or complementary to a sequence encoding a sphingosine kinase variant or derivative, homologue, analogue, chemical equivalent or mimetic of said variant which variant comprises a mutation in a region defined by amino acid 16-153 or functionally equivalent region wherein said variant exhibits ablated or reduced catalytic activity relative to wild-type sphingosine kinase.
(ii) An isolated nucleic acid molecule or derivative or equivalent thereof comprising a nucleotide sequence encoding or complementary to a sequence encoding a human sphingosine kinase variant or derivative, homologue, analogue, chemical equivalent or mimetic of said variant which variant comprises a mutation in a region defined by amino acid 16-153 or functionally equivalent region wherein said variant exhibits ablated or reduced catalytic activity relative to wild-type human sphingosine kinase.
(iii) An isolated nucleic acid molecule or derivative or equivalent thereof comprising a nucleotide sequence encoding or complementary to a sequence encoding a human sphingosine kinase variant or derivative, homologue, analogue, chemical equivalent or mimetic of said variant, which variant comprises an amino acid sequence with a single or multiple amino acid substitution, addition and/or deletion in a region defined by amino acid 16-153 or functionally equivalent region wherein said variant exhibits ablated or reduced catalytic activity relative to wild-type sphingosine kinase.
(iv) An isolated nucleic acid molecule or derivative or equivalent thereof comprising a nucleotide sequence encoding or complementary to a sequence encoding a human sphingosine kinase variant or derivative, homologue, analogue, chemical equivalent or mimetic of said variant, which variant comprises an amino acid sequence with a single or multiple amino acid substitution, addition and/or deletion in a region defined by amino acid 70-90 or functionally equivalent region wherein said variant exhibits ablated or reduced catalytic activity relative to wild-type sphingosine kinase.
(v) An isolated nucleic acid molecule or derivative or equivalent thereof comprising a nucleotide sequence encoding or complementary to a sequence encoding a human sphingosine kinase variant or derivative, homologue, analogue, chemical equivalent or mimetic of said variant, which variant comprises an amino acid sequence with a single or multiple amino acid substitution, addition and/or deletion in a region defined by amino acid 79-84 or functionally equivalent region wherein said variant exhibits ablated or reduced catalytic activity relative to wild-type sphingosine kinase.
(vi) An isolated nucleic acid molecule or derivative or equivalent thereof comprising a nucleotide sequence encoding or complementary to a sequence encoding a sphingosine kinase variant or a derivative, homologue, analogue, chemical equivalent or mimetic of said variant comprising one or more of the amino acid substitutions selected from the following list:
   (a) G82D
   (b) G82A
   (c) G26D
   (d) S79D
   (e) G80D
   (f) K103A
   (g) G111D
   (h) G113D
   (i) G26A
   (K27A
   (k) K29A
   (l) S79A
   (m) G80A
   (n) K103R
   (o) G111A
(vii) An isolated nucleic acid molecule or derivative or analogue thereof comprising a nucleotide sequence encoding or complementary to a sequence encoding a sphingosine kinase variant or derivative, homologue, analogue, chemical equivalent or mimetic of said variant which variant comprises a mutation in an ATP binding site region or functionally equivalent region wherein said variant exhibits ablated or reduced catalytic activity relative to wild-type sphingosine kinase.

The nucleic acid molecule of the subject invention may be ligated to an expression vector capable of expression in a prokaryotic cell (eg. E. Coli) or a eukaryotic cell (eg. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of the variant sphingosine kinase encompassed by the present invention.

The variant sphingosine kinase molecule of the present invention may be derived from natural or recombinant sources or may be chemically synthesised. Methods for producing these molecules would be well known to those skilled in the art.

In addition to facilitating the synthesis of sphingosine kinase variants, per se, identification of the mechanism of functioning of the sphingosine kinase variants of the present invention permits the design of methodology for ablating or decreasing the catalytic activity of wild-type sphingosine kinase proteins. For example, contacting wild-type sphingosine kinase proteins with an agent which bind to or otherwise associate with the region defined by amino acids 16-153 or functionally equivalent region is possible to effectively convert a wild-type sphingosine kinase protein to a catalytically inactive variant. In another aspect, said agent could bind to or otherwise associate with the sphingosine kinase ATP binding site.

Without limiting the present invention in any way, it is thought that baseline sphingosine kinase activity is a constitutive property of all wild-type sphingosine kinase proteins. In order to activate this molecule, though, one has to increase the Vmax of the enzyme (eg., by transporting more of the enzyme to the location at which it is required or by altering its function post-translationally). This requires the functioning of another molecule or class of molecules (such as a protein or lipid) to associate with the subject sphingosine kinase, these molecules hypothetically being termed FOSK(s) (Friends of Sphingosine Kinase). Reference to “FOSK” should. be understood to include reference to sphingosine kinase interacting molecules (SKIMS). The G82D sphingosine kinase variant, for example, is thought to bind a FOSK molecule thereby preventing it from activating wild-type sphingosine kinase.

Accordingly, the present invention provides not only sphingosine kinase variants, per se, as potential drugs, but provides a mechanism for further drug development. For example, a small molecule that binds to the region which is the subject of mutation in the sphingosine kinase variant molecule would be expected to convert a wild-type sphingosine kinase protein to an inhibitor that not only loses its baseline sphingosine kinase activity but also causes its conversion into a dominant negative sphingosine kinase variant, ie., both levels of sphingosine kinase activity are eliminated. Alternatively, screening for an agent which prevents interaction of the wild-type sphingosine kinase with a FOSK would reproduce the dominant negative phenotype while not disturbing baseline sphingosine kinase activity but only inhibiting activation. This is a potentially desirable scenario where some sphingosine kinase activity is required for cellular survival.

Modulation of the activity between sphingosine kinase may therefore be achieved by any one of a number of techniques including, but not limited to:
(i) introducing into a cell a proteinaceous or non-proteinaceous molecule which antagonises the interaction between a FOSK and sphingosine kinase.
(ii) introducing into a cell a proteinaceous or non-proteinaceous molecule which interacts with at least part of the region of wild type sphingosine kinase which is the subject mutation in the variants described herein.

Reference to “agent” should therefore be understood as a reference to any proteinaceous or non-proteinaceous molecule which modulates the interaction of sphingosine kinase with a FOSK or interacts with at least part of the region defined by amino acids 16-153 of sphingosine kinase and includes, for example, the molecules detailed in points (i)-(ii), above. The subject agent may be linked, bound or otherwise associated with any proteinaceous or non-proteinaceous molecule. For example, it may be associated with a molecule which permits its targeting to a localised region.

Accordingly, by administering said agent intracellularly, endogenously produced wild-type sphingosine kinase could be both inactivated in terms of its baseline activity and induced to function as a dominant negative sphingosine kinase molecule wherein the agent-associated wild-type sphingosine kinase functions to ablate or decrease activation of other non-agent associated wild-type sphingosine kinase molecules. Alternatively, since in some instances it is necessary to maintain intracellular baseline sphingosine kinase activity, the agent could be associated with wild-type sphingosine kinase extracellularly and the agent-sphingosine kinase complex could then be administered intracellularly. These complexes (being a variant sphingosine kinase within the context of the present invention) would then act to ablate or decrease wild-type sphingosine kinase activation without significantly modulating baseline activity of endogenously produced wild-type sphingosine kinase proteins. The design and generation of these molecules provides a unique and previously unavailable mechanism for modulating activity of the sphingosine kinase signalling path. Specifically, whereas previously utilised chemical inhibitors such as N′N-dimethylsphingosine totally eliminate sphingosine kinase functioning, the variants of the present invention can be administered such as to only reduce or eliminate activation of wild-type sphingosine kinase without disturbing baseline functioning.

The subject agent may be any proteinaceous or non-proteinaceous molecule derived from natural, recombinant or synthetic sources including fusion proteins or following, for example, natural product screening and which achieves the object of the present invention. Synthetic sources of said agent include for example chemically synthesised molecules. In other examples, phage display libraries can be screened for peptides while chemical libraries can be screened for existing small molecules. Rational drug design/structure based design can be achieved by performing crystallisation, further analysing the ATP binding site and fitting molecules into that site by design.

By way of example, diversity libraries, such as random combinatorial peptide or nonpeptide libraries can be screened. Many publically or commercially available libraries can be used such as chemically synthesized libraries, recombinant (e.g., phage display libraries) and in vitro translation-based libraries.

Examples of chemically synthesized libraries are described in Fodor et al., (1991); Houghten et al., (1991); Lam et al., (1991); Medynski., (1994); Gallop et al., (1994); Ohlmeyer et al., (1993); Erb et al., (1994); Houghten et al., (1992); Jayawickreme et al., (1994); Salmon et al., (1993); International Patent Publication No. WO 93/20242; and Brenner and Lerner., (1992).

Examples of phage display libraries are described by Scott and Smith., (1990); Devlin et al., (1990); Christian R. B et al., (1992); Lenstra., (1992); Kay et al., (1993) and International Patent Publication No. WO 94/18318.

In vitro translation-based libraries include but are not limited to those described in Mattheakis et al., (1994).

Screening the libraries can be accomplished by any of a variety of commonly known methods. See Parmley and Smith., (1989); Scott and Smith., (1990); Fowlkes et al., (1992); Oldenburg et al., (1992); Yu et al., (1994); Staudt et al., (1988); Bock et al., (1992); Tuerk et al., (1992); Ellington et al., (1992); U.S. Pat. No. 5,096,815, U.S. Pat. No. 5,223,409 and U.S. Pat. No. 5,198,346; Rebar and Pabo., (1993); and International Patent Publication No. WO 94/18318.

The present invention should therefore also be understood to extend to a method of screening for agents which modulate the interaction between sphingosine kinase and a FOSK molecule. This could be achieved, for example, by utilising cell based assays which can monitor sphingosine kinase activation. Accordingly, the present invention provides a mechanism of screening for agents which utilise one of a variety of methods of inhibiting sphingosine kinase. For example, agents which totally ablate both levels of sphingosine kinase activity can be screened for in addition to molecules which only inhibit sphingosine kinase activation (for example by inhibiting the interaction between sphingosine kinase and a FOSK).

Screening for the modulatory agents hereinbefore defined can be achieved by any one of several suitable methods including, but in no way limited to, contacting a cell comprising sphingonsine kinase (separately or together with FOSK) with an agent and screening for the modulation of sphingosine kinase/FOSK functional activity or modulation of the activity or expression of a downstream sphingosine kinase or FOSK cellular target. Detecting such modulation can be achieved utilising techniques such as Western blotting, electrophoretic mobility shift assays and/or the readout of reporters of sphingosine kinase or FOSK activity such as luciferases, CAT and the like.

It should be understood that the sphingosine kinase or FOSK protein may be naturally occurring in the cell which is the subject of testing or the genes encoding them may have been transfected into a host cell for the purpose of testing. Further, the naturally occurring or transfected gene may be constitutively expressed—thereby providing a model useful for, inter alia, screening for agents which down-regulate sphingosine kinase FOSK interactivity or the gene may require activation—thereby providing a model useful for, inter alia, screening for agents which modulate sphingosine kinase/FOSK interactivity under certain stimulatory conditions. Further, to the extent that a sphingosine kinase nucleic acid molecule is transfected into a cell, that molecule may comprise the entire sphingosine kinase gene or it may merely comprise a portion of the gene such as the FOSK binding portion.

In another example, the subject of detection could be a downstream sphingosine kinase regulatory target, rather than sphingosine kinase itself. Yet another example includes sphingosine kinase binding sites ligated to a minimal reporter. For example, modulation of sphingosine kinase/FOSK interactivity can be detected by screening for the modulation of the downstream signalling components of a TNF stimulated cell. This is an example of a system where modulation of the molecules which sphingosine kinase and FOSK regulate the activity of, are monitored.

Accordingly, another aspect of the present invention provides a method for detecting an agent capable of modulating the interaction of FOSK with sphingosine kinase or its functional equivalent or derivative thereof said method comprising contacting a cell or extract thereof containing said sphingosine kinase and FOSK or its functional equivalent or derivative with a putative agent and detecting an altered expression phenotype associated with said interaction.

Reference to “sphingosine kinase” and “FOSK” should be understood as a reference to either the sphingosine kinase or FOSK expression product or to a portion or fragment of the sphingosine kinase or FOSK molecule, such as the FOSK region defined by amino acids 16-153 of the sphingosine kinase protein. In this regard, the sphingosine kinase or FOSK expression product is expressed in a cell. The cell may be a host cell which has been transfected with the sphingosine kinase or FOSK nucleic acid molecule or it may be a cell which naturally contains the sphingosine kinase gene. Reference to “extract thereof” should be understood as a reference to a cell free transcription system.

Reference to detecting an “altered expression phenotype associated with said interaction” should be understood as the detection of cellular changes associated with modulation of the interaction of sphingosine kinase with FOSK. These may be detectable, for example, as intracellular changes or changes observable extracellularly. For example, this includes, but is not limited to, detecting changes in downstream product levels or activities.

In yet another aspect the present invention provides a method for detecting an agent capable of binding or otherwise associating with the sphingosine kinase region defined by amino acids 16-153 or functional equivalent or derivative thereof said method comprising contacting a cell containing said amino acid region or functional equivalent or derivative thereof with a putative agent and detecting an altered expression phenotype associated with modulation of the function of sphingosine kinase or its functional equivalent or derivative.

Preferably, said region is defined by amino acids 70-90 and even more preferably 79-84.

Reference to “sphingosine kinase binding site” should be understood as a reference to the sphingosine kinase region defined by amino acids 16-153, preferably 70-90 and even more preferably 79-84.

In addition to screening for agents which modulate the interaction of FOSK and sphingosine kinase utilising function based assays of the type described above, the identification of the functionally active region of sphingosine kinase also facilitates the screening, analysis, rational design and/or modification of agents for modulating either the interaction of FOSK and sphingosine kinase or the activity of sphingosine kinase based on analysis of the physical interaction of a putative agent or lead compound with the subject region.

Specifically, knowledge of the nature and location of this site now facilitates analysis of the tertiary structure of sphingosine kinase, in terms of the structure of the binding site, by techniques such as X-ray crystallography.

Accordingly, another aspect of the present invention is directed to a method for analysing, designing and/or modifying an agent capable of interacting with the sphingosine kinase region defined by amino acids 16-153 or derivative thereof and modulating at least one functional activity associated with said sphingosine kinase said method comprising contacting said sphingosine kinase or derivative thereof with a putative agent and assessing the degree of interactive complementarity of said agent with said binding site.

Preferably, said region is defined by amino acids 70-90 and even more preferably 79-84.

It should be understood that the sphingosine kinase which is contacted with the putative agent for evaluation of interactive complementarity may be recombinantly produced. However, it should also be understood that the subject sphingosine kinase may take the form of an image based on the binding site structure which has been elucidated, such as an electron density map, molecular models (including, but not limited to, stick, ball and stick, space filling or surface representation models) or other digital or non-digital surface representation models or image, which facilitates the analysis of sphingosine kinase site: agent interactions utilising techniques and software which would be known to those of skill in the art. For example, interaction analyses can be performed utilising techniques such as Biacore real-time analysis of on and off-rates and dissociation constants for binding of ligands (Gardsvoll et al, 1999; Hoyer-Hansen et al, 1997; Ploug, 1998; Ploug et al, 1994; 1995; 1998) and NMR perturbation studies (Stephens et al, 1992).

Reference to “assessing the degree of interactive complementarity” of an agent with the subject sphingosine kinase binding site should be understood as a reference to elucidating any feature of interest including, but not limited to, the nature and/or degree of interaction between the subject sphingosine kinase binding site and an agent of interest. As detailed above, any suitable technique can be utilised. Such techniques would be known to the person of skill in the art and can be utilized in this regard. In terms of the nature of the subject interaction, it may be desirable to assess the types of interactive mechanisms which occur between specific residues of any given agent and those of the sphingosine kinase binding site (for example, peptide bonding or formation of hydrogen bonds, ionic bonds, van der Waals forces, etc.) and/or their relative strengths. It may also be desirable to assess the degree of interaction which occurs between an agent of interest and the subject sphingosine kinase binding site. For example, by analysing the location of actual sites of interaction between the subject agent and sphingosine kinase binding site it is possible to determine the quality of fit of the agent into this region of the sphingosine kinase binding site and the relative strength and stability of that binding interaction. For example, if it is the object that sphingosine kinase binding site functioning be blocked, an agent which interacts with the sphingosine kinase binding site such that it blocks or otherwise hinders (for example, sterically hinders or chemically or electrostatically repels) FOSK interaction or down-regulates sphingosine kinase activity will be sought. The form of association which is required in relation to modulating sphingosine kinase functioning may not involve the formation of any chemical interactive bonding mechanism, as this is traditionally understood, but may involve a non-bonding mechanism such as the proximal location of a region of the agent relative to the subject binding region of the sphingosine kinase binding site, for example, to effect steric hindrance with respect to the binding of an activating molecule. Where the interaction takes the form of hindrance or the creation of other repulsive forces, this should nevertheless be understood as a form of “interaction” despite the lack of formation of any of the traditional forms of bonding mechanisms.

It should also be understood that the sphingosine kinase binding site which is utilised either in a physical form or as an image, as hereinbefore discussed, to assess the interactive complementarity of a putative agent may be a naturally occurring form of the sphingosine kinase binding site or it may be a derivative, homologue, analogue, mutant, fragment or equivalent thereof. The derivative, homologue, analogue, mutant, fragment or equivalent thereof may take either a physical or non-physical (such as an image) form.

The determination of sphingosine kinase binding regions facilitates determination of the three dimensional structure of the sphingosine kinase binding site and the identification and/or rational modification and design of agents which can be used to modulate FOSK binding or sphingosine kinase functioning.

Without limiting the application of the present invention in any way, the method of the present invention facilitates the analysis, design and/or modification of agents capable of interacting with the sphingosine kinase site defined by amino acids 16-153. In this regard, reference to “analysis, design and/or modification” of an agent should be understood in its broadest sense to include:
(i) Randomly screening (for example, utilising routine high-throughput screening technology) to identify agents which exhibit some modulatory capacity with respect to sphingosine kinase functional activity and/or FOSK binding and then analysing the precise nature and magnitude of the agent's modulatory capacity utilising the method of this aspect of the present invention. In this regard, existing crystals could be soaked with said agents or co-crystalisation could be performed. A combination of modelling and synthetic modification of the local compound together with mutagenesis of the sphingosine kinase binding site could then be performed for example. In screening for agents which may modulate activity, standard methods of phage display and also combinatorial chemistry may be utilised (Goodson et al., 1994; Terrett., 2000). Such interaction studies can also be furthered utilising techniques such as the Biacore analysis and NMR perturbation studies. Such agents are often commonly referred to as “lead” agents in terms of the random screening of proteinaceous or non-proteinaceous molecules for their capacity to function either agonistically or antagonistically. Further, for example, binding affinity and specificity could be enhanced by modifying lead agents to maximise interactions with the sphingosine kinase binding site. Such analyses would facilitate the selection of agents which are the most suitable for a given purpose. In this way, the selection step is based not only on in vitro data but also on a technical analysis of sites of agent: sphingosine kinase interaction in terms of their frequency, stability and suitability for a given purpose. For example, such analysis may reveal that what appears to be an acceptable in vitro activity in respect of a randomly identified agent is in fact induced by a highly unstable interaction due to the presence of proximally located agent: sphingosine kinase sites which exhibit significant repulsive forces thereby de-stabilising the overall interaction between the agent and the sphingosine kinase. This would then facilitate the selection of another prospective lead compound, exhibiting an equivalent degree of in vitro activity, but which agent does not, upon further analysis, involve the existence of such de-stabilising repulsive forces.

Screening for the modulatory agents herein defined can be achieved by any one of several suitable methods, including in silico methods, which would be well known to those of skill in the art and which are, for example, routinely used to randomly screen proteinaceous and non-proteinaceous molecules for the purpose of identifying lead compounds.

These methods provide a mechanism for performing high throughput screening of putative modulatory agents such as the proteinaceous or non-proteinaceous agents comprising synthetic, recombinant, chemical and natural libraries.
(ii) The candidate or lead agent (for example, the agent identified in accordance with the methodology described in relation to point (i)) could be modified in order to maximise desired interactions (for example, binding affinity to specificity) with the sphingosine kinase and to minimise undesirable interactions (such as repulsive or otherwise de-stabilising interactions).
   Methods of modification of a candidate or lead agent in accordance with the purpose as defined herein would be well known to those of skill in the art. For example, a molecular replacement program such as Amore (Navaza, 1994) may be utilised in this regard. The method of the present invention also facilitates the mutagenesis of known signal inducing agents in order to ablate or improve signalling activity.
(iii) In addition to analysing fit and/or structurally modifying existing molecules, the method of the present invention also facilitates the rational design and synthesis of an agent, such as an agonistic or antagonistic agent, based on theoretically modelling an agent exhibiting the desired sphingosine kinase binding site interactive structural features followed by the synthesis and testing of the subject agent.

It should be understood that any one or more of applications (i)-(iii) above, may be utilised in identifying a particular agent.

In a related aspect, the present invention should be understood to extend to the agents identified utilising any of the methods hereinbefore defined. In this regard, reference to an agent should be understood as a reference to any proteinaceous or non-proteinaceous molecule which modulates at least one sphingosine kinase functional activity.

Without limiting the theory or mode of action of the present invention, sphingosine kinase is a key regulatory enzyme in the activity of the sphingosine kinase signalling pathway.

By “sphingosine kinase signalling pathway” is meant a signalling pathway which utilises one or both of sphingosine kinase and/or sphingosine-1-phosphate. It is thought that a sphingosine kinase signalling pathway cascade may take the form of:
(i) the generation of ceramide from sphingomyelin via S. Mase activity, said ceramide being converted to sphingosine;
(ii) sphingosine-1-phosphate generation by stimulation of sphingosine kinase; and
(iii) the activation of MEK/ERK and nuclear translocation of NF-κB downstream from Sphingosine-1-phosphate generation.

The sphingosine kinase signaling pathway is known to regulate cellular activities such as those which lead to inflammation, apoptosis and cell proliferation. For example, upregulation of the production of inflammatory mediators such as cytokines, chemokines, eNOS and upregulation of adhesion molecule expression. Said upregulation may be induced by a number of stimuli including, for example, inflammatory cytokines such as tumour necrosis factor-α (TNF-α) and interleukin-1 (IL-1), endotoxin, oxidised or modified lipids, radiation or tissue injury.

The generation of variant sphingosine kinase molecules now provides additional molecules for use in the prophylactic and therapeutic treatment of diseases characterised by unwanted cellular activity, which activity is either directly or indirectly modulated via activity of the sphingosine kinase signalling pathway. Examples of diseases involving unwanted sphingosine kinase regulated cellular activity include inflammatory conditions (eg., rheumatoid arthritis, inflammatory bowel disease), neoplastic conditions (eg., solid cancers), asthma, atherosclerosis, meningitis, multiple sclerosis and septic shock. The variants of the present invention may also facilitate the provision of chronic treatment in relation to disease conditions such as atherosclerosis, osteoarthritis and other degenerative diseases in which inflammation plays a role.

Accordingly, the present invention contemplates therapeutic and prophylactic uses of variant sphingosine kinase molecules for the regulation of cellular functional activity, such as for example, regulation of inflammation. In this regard, the variant molecules which may be used in therapy and prophylaxis include mutated sphingosine kinase expression product, nucleic acid molecules encoding mutated sphingosine kinase expression product, sphingosine kinase-agent complexes as hereinbefore defined or an agent, per se, which is proposed to be administered to a subject for the purpose of its intracellular complexation with wild-type sphingosine kinase for the purpose of converting a wild-type molecule to a variant sphingosine kinase molecule. For ease of reference and in accordance with the definitions provided earlier, it should be understood that the phrase “sphingosine kinase variant” includes reference to mutated sphingosine kinase proteins, nucleic acid molecules encoding said proteins and sphingosine kinase-agent complexes while reference to “agent” is intended to refer to an agent which, when contacted with a sphingosine kinase protein (such as a wild-type protein) will render the protein a variant within the context of the present invention.

Accordingly, another aspect of the present invention contemplates a method of modulating cellular functional activity in a mammal said method comprising administering to said mammal an effective amount of a sphingosine kinase variant or agent as hereinbefore defined for a time and under conditions sufficient to inhibit, reduce or otherwise down-regulate at least one functional activity of wild-type sphingosine kinase.

Preferably said functional activity is down-regulation of wild-type sphingosine kinase baseline activity and/or prevention of wild-type sphingosine kinase activation.

Reference to “modulating cellular functional activity” is a reference to up-regulating, down-regulating or otherwise altering any one or more of the activities which a cell is capable of performing such as, but not limited to, one or more of chemokine production, cytokine production, nitric oxide synthetase, adhesion molecule expression and production of other inflammatory modulators.

Administration of the variant sphingosine kinase or agent, in the form of a pharmaceutical composition, may be performed by any convenient means. Variant sphingosine kinase or agent of the pharmaceutical composition are contemplated to exhibit therapeutic activity when administered in an amount which depends on the particular case. The variation depends, for example, on the human or animal and the sphingosine kinase or agent chosen. A broad range of doses may be applicable. Considering a patient, for example, from about 0.1 mg to about 1 mg of sphingosine kinase or agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation. The variant sphingosine kinase or agent may be administered in a convenient manner such as by the oral, intravenous (where water soluble), intranasal, intraperitoneal, intramuscular, subcutaneous, intradermal or suppository routes or implanting (e.g. using slow release molecules). With particular reference to use of variant sphingosine kinase or agent, these molecules may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

A further aspect of the present invention relates to the use of the invention in relation to mammalian disease conditions. For example, the present invention is particularly useful, but in no way limited to, use in therapeutically or prophylactically treating inflammatory diseases, neoplastic conditions and degenerative diseases.

Accordingly, another aspect of the present invention relates to the treatment and/or prophylaxis of a condition in a mammal, which condition is characterised by aberrant, unwanted or otherwise inappropriate cellular activity, said method comprising administering to said mammal an effective amount of a sphingosine kinase variant or agent as hereinbefore defined for a time and under conditions sufficient to inhibit, reduce or otherwise down-regulate at least one functional activity of wild-type sphingosine kinase wherein said down-regulation results in modulation of cellular functional activity.

Preferably said functional activity is down-regulation of baseline wild-type sphingosine kinase activity and/or prevention of wild-type sphingosine kinase activation.

Reference to “aberrant, unwanted or otherwise inappropriate” cellular activity should be understood as a reference to overactive cellular activity, underactive cellular activity or physiologically normal cellular activity which is inappropriate or otherwise unwanted.

The subject of the treatment or prophylaxis is generally a mammal such as but not limited to human, primate, livestock animal (eg. sheep, cow, horse, donkey, pig), companion animal (eg. dog, cat), laboratory test animal (eg. mouse, rabbit, rat, guinea pig hamster), captive wild animal (eg. fox, deer). Preferably the mammal is human or primate. Most preferably the mammal is a human.

Reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis including amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

An “effective amount” means an amount necessary at least partly to attain the desired immune response, or to delay the onset or inhibit progression or halt altogether, the onset or progression of a particular condition of the individual to be treated, the taxonomic group of individual to be treated, the degree of protection desired, the formulation of the vaccine, the assessment of the medical situation, and other relevant factors. It is expected that the amount will fall in a relatively broad range that can be determined through routine trials.

A further aspect of the present invention relates to the use of a sphingosine kinase variant or agent as hereinbefore defined in the manufacture of a medicament for the modulation of cellular functional activity.

Another aspect of the present invention relates to a sphingosine kinase variant or agent as hereinbefore defined for use in modulating cellular functional activity.

In a related aspect of the present invention, the mammal undergoing treatment may be a human or an animal in need of therapeutic or prophylactic treatment.

In accordance with these methods, the molecules defined in accordance with the present invention may be coadministered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another further aspect the present invention contemplates a pharmaceutical composition comprising a sphingosine kinase variant or agent as hereinbefore defined together with one or more pharmaceutically acceptable carriers and/or diluents. The sphingosine kinase variant and agent are referred to as the active ingredients.

The pharmaceutical forms suitable for injectable use include sterile aqueous solutions (where water soluble) and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as licithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assailable edible carrier, or they may be enclosed in hard or soft shell gelatin capsule, or they may be compressed into tablets, or they may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release, preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

The pharmaceutical composition may also comprise genetic molecules such as a vector capable of transfecting target cells where the vector carries a nucleic acid molecule capable of expressing a sphingosine kinase variant or agent. The vector may, for example, be a viral vector.

Further features of the present invention are more fully described in the following non-limiting examples.

### EXAMPLE 1

### Materials and Methods

Materials—D-erythro-Sphingosine and sphingosine-1-phosphate were purchased from Biomol Research Laboratories Inc. (Plymouth Meeting, Pa.). ATP and phorbol 12-myristate 13-acetate (PMA) were from Sigma. [γ³²P] ATP and ³²P-phosphoric acid were purchased from Geneworks (Adelaide, South Australia), [choline-methyl-¹⁴C]sphingomyelin from NEN (Boston, Mass.), TNFα from R&D Systems Inc. (Minneapolis, Minn.). Interleukin 1 (IL-1) was a gift from Synergin (Bolder, Colo.).

Cell Culture and Transfection—Human embryonic kidney cells (HEK293T, ATCC CRL-1573) cells were cultured on Dulbecco's modified Eagle's medium (DMEM; CSL Biosciences, Parkville, Austrlia) containing 10% fetal calf serum, 2 mM glutamine, 0.2% (w/v) sodium bicarbonate, penicillin (1.2 mg/ml), and gentamycin (1.6 mg/ml). Transfections were performed using the calcium phosphate precipitation method (Graham & van der Eb, 1973). Cells were harvested and lysed by sonication (2 watts for 30 s at 4° C.) in lysis buffer containing 50 mM Tris/HCl (pH 7.4), 10% glycerol, 0.05% Triton X-100, 150 mM NaCl, 1 mM dithiothreitol, 2 mM Na₃VO₄ 10 mM NaF, and 1 mM EDTA. Protein concentrations in cell homogenates were determined with either the Coomassie Brillant Blue (Sigma) or Bichinchoninic acid (Pierce) reagents using BSA as standard.

Enzyme Assays—Sphingosine kinase activity was determined using D-erythro-sphingosine and [γ³²P]ATP as substrates, as described previously (Pitson et al., in press). Neutral sphingomyelinase activity was determined using [choline-methyl-¹⁴C]sphingomyelin as substrate, essentially as previously described (Wiegmann et al., 1994). Briefly, whole cell lysates, prepared as described above, were added to an equal volume of 100 mM Tris/HCl buffer (pH 7.4) containing 0.2% Triton X-100, 10 mM MgCl₂ and [choline-methyl-¹⁴C]sphingomyelin (50,000 cpm/assay) and incubated at 37° C. for 60 min. Radioactive phosphorylcholine produced was then extracted with chloroform/methanol (2:1, v/v) and quantified in the aqueous phase by scientillation counting. The measurement of PKC activity in situ was performed as described previously (Xia et al., 1996). Briefly, cells were seeded in 24-well plates and maintained in culture medium until 70-80% confluent. After the indicated treatments, the cells were washed with DMEM and placed in 60 μl of buffered salt solution (137 mM NaCl, 5.4 mM KCl, 0.3 mM Na₂HPO₄, 0.4 mM KH₂PO₄, 5.5 mM glucose, and 20 mM HEPES) supplemented with 50 μg/ml digitonin, 10 mM MgCl₂, 25 mM β-glycerophosphate, and 10 μM [γ³²P]ATP (5000 cpm/pmol). A PKC-specific peptide substrate (RKRTLRRL) was then added (to 2001) in the presence of 5 mM EGTA and 2.5 mM CaCl₂. After a 10 min incubation at 30° C., the kinase reaction was terminated by the addition of 20 μl of 25% (w/v) trichloroacetic acid. Aliquots (65 μl) of the acidified reaction mixtures were spotted on phosphocellulose papers (Whatman P-81) and washed three times with 75 mM phosphoric acid and once with 75 mM sodium phosphate (pH 7.5). The PKC-dependent phosphorylated peptide substrate bound to the filter was quantified by scintillation counting.

Western Blotting—SDS-PAGE was performed on cell lysates according to the method of Laemmli [29] using 12% acrylamide gels. Proteins were blotted to nitrocellulose and the membranes blocked overnight at 4° C. in PBS containing 5% skim milk and 0.1% Triston X-100. Sphingosine kinase expression levels were analysed with the M2 anti-FLAG antibody (Sigma). ERK activation in response to agonists was followed in cells serum-starved for 4 h using anti-ERK1/2 (Zymed, San Francisco, Calif.) and anti-phospho-ERK1/2 (Promega, Madison, Wis.) antibodies. Immunocomplexes were detected after conjugation to either HRP anti-mouse (Pierce) or anti-rabbit (Selinus/AMRAD, Melbourne, Australia) IgG using an enhanced chemiluminescence kit (Amersham).

### Mutagenesis of the SK-1 Sequence

The SK-1 cDNA (as described in Pitson et al. 2000a) was cloned into pALTER (promega Inc., Madison, Wis.) site directed mutagenesis vector. Single-stranded DNA was prepared and used as template for oligonucleotide directed mutagenesis as detailed in the manufacturer's protocol. The mutagenic oligonucleotide (5′CTG GAG ACG ATC TGA TGC AC) [<400>1] was designed to generate the G82D mutant, substitution of the glycine at position 82 to aspartic acid. The mutant was sequenced to verify incorporation of the desired modification.

Expression of the G82D cDNA

The G82D mutant cDNA was sub-cloned into pcDNA3 (Invitrogen Corp., San Diego Calif.). The expression construct was transfected by calcilum phosphate precipitation into HEK293T cells.

### Transformation Assay

For focus formation assay, low passage NIH 3T3 cells were transfected with the V12 mutant H-ras, v-SRC (gifts from Dr. Julian Downward²), SphK, G82D mutant SphK expression vectors or empty vector using Lipofectamine Plus as described above. Two days later, the transfected cells were split to 6-well plates. After reaching confluence, they were kept for two weeks in DMEM containing 5% calf serum. The foci were visualized and scored after staining with 0.5% crystal violet. For soft agar assay, suspensions of 1×10⁴ cells from the stable transfected pools in a growth medium containing 0.33% agar were overlaid onto 0.6% agar gel in the absence or presence of DMS at various concentrations. After 14-days incubation colonies were stained with 0.1 mg/ml MTT and those greater than 0.1 mm in diameter were scored as positive.

### EXAMPLE 2

### Results

A mutant of sphingosine kinase that is inactive in its capacity to phosphorylate sphingosine to sphingosine-1-phosphate (S1P), the molecule that mediates the biologically relevant functions of sphingosine kinase, has been designed and made. This mutant was made by site directed mutagenesis of a putative ATP binding site (G in position 82 to aspartic acid ‘G82D’), thus rendering the sphingosine kinase catalytically inactive

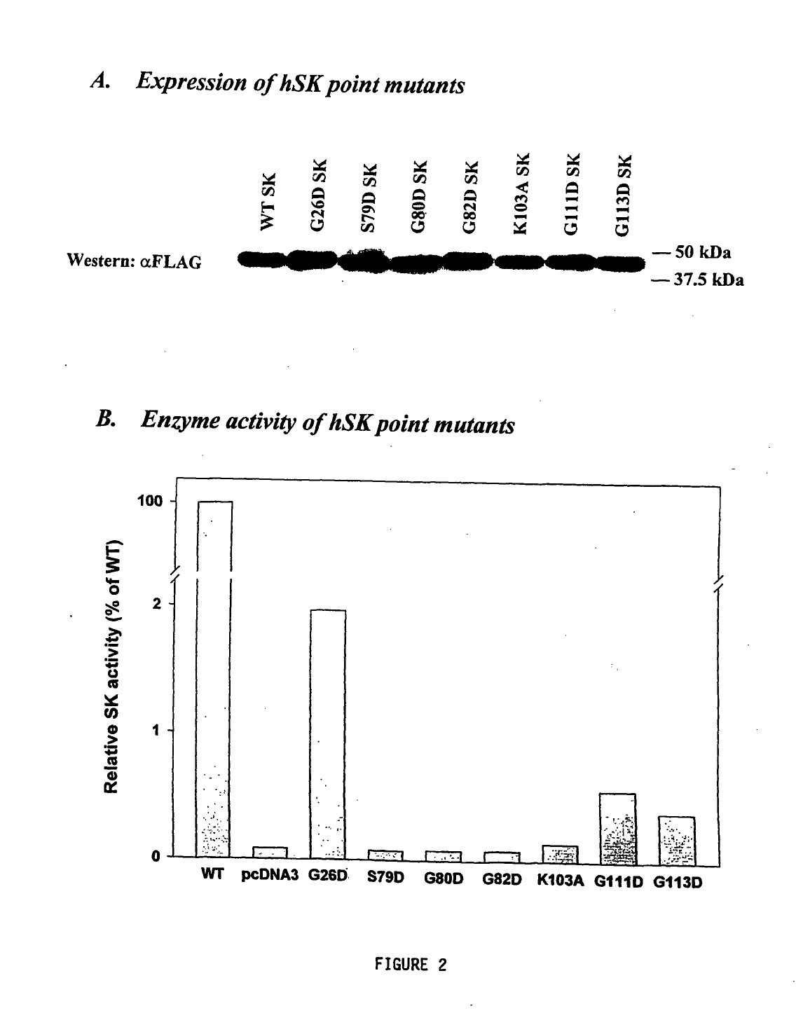

G82DSK is well expressed as seen in Western blots (FIG. 2) of the FLAG tagged transfectants and is correctly folded as judged by binding to calmodulin (data not shown).

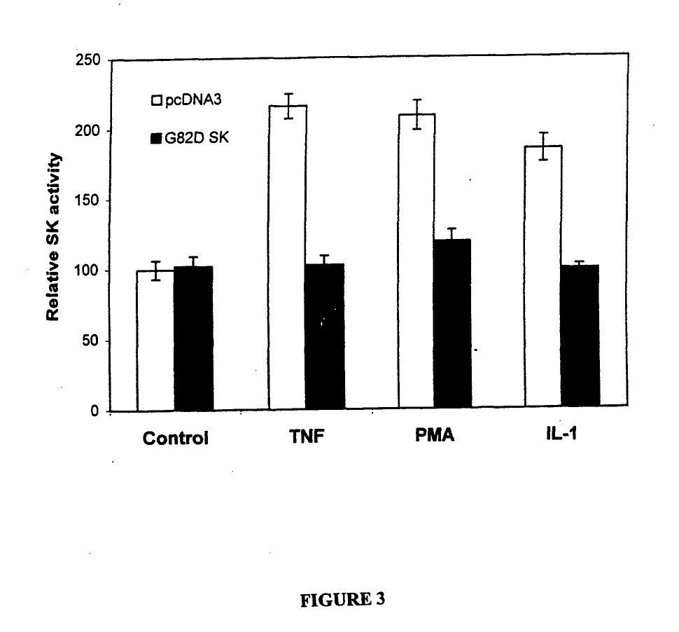

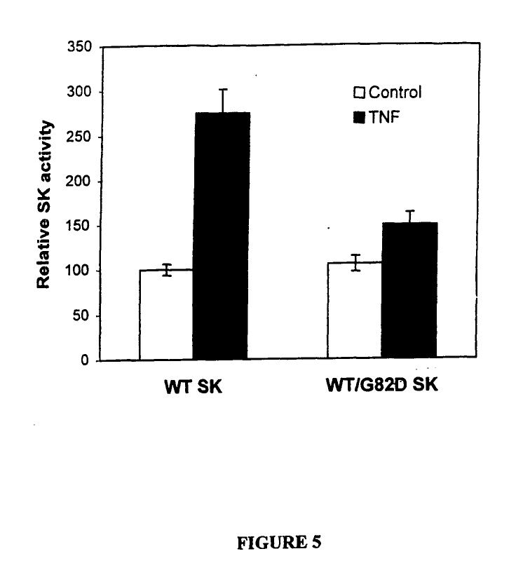

The G82DSK by itself has no sphingosine kinase activity and does not suppress endogenous baseline sphingosine kinase activity (FIG. 2), however it totally suppresses the increases in sphingosine kinase activity seen after treatment of cells with activating agents such as TNF, IL-1 and PMA (FIGS. 3 & 4). This ‘duality’ of function is also seen in cells that overexpress sphingosine kinase: the overexpressed baseline levels are not altered, but activation is decreased (FIG. 5). The extent of prevention is likely to depend on the molar ratio of sphingosine kinase:G82DSK.

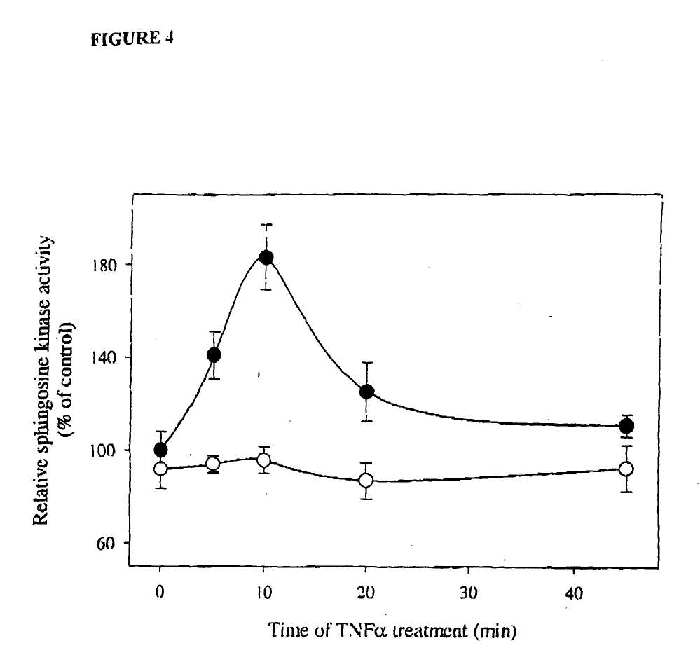

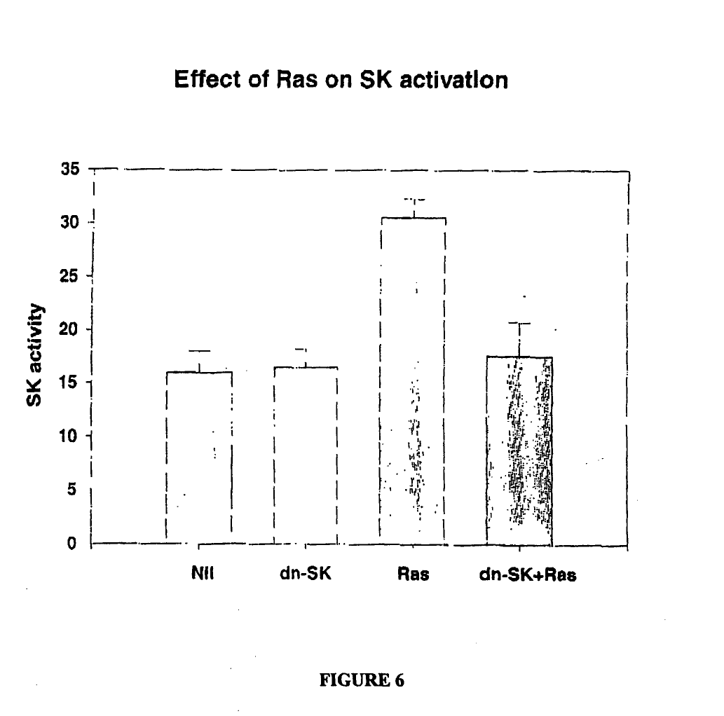

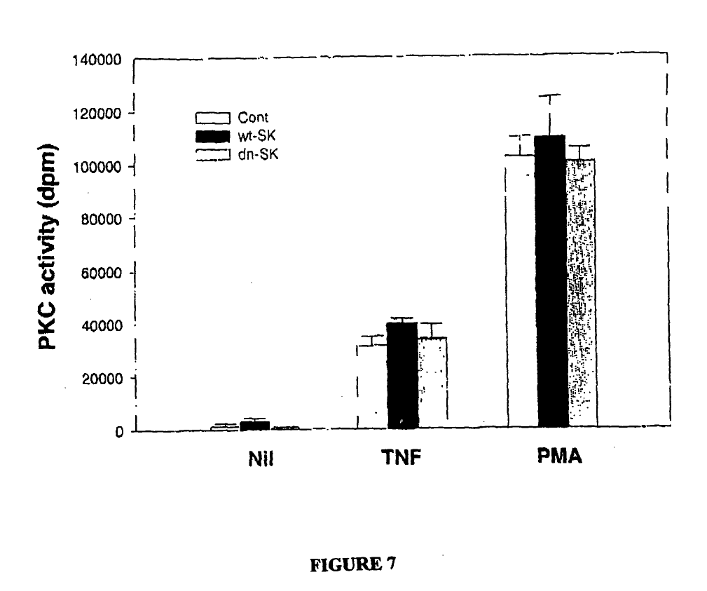

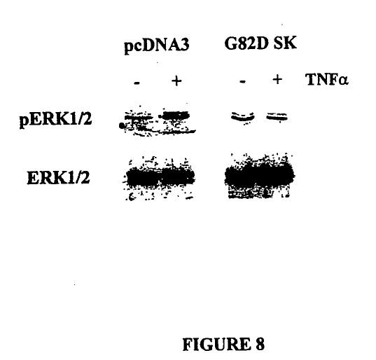

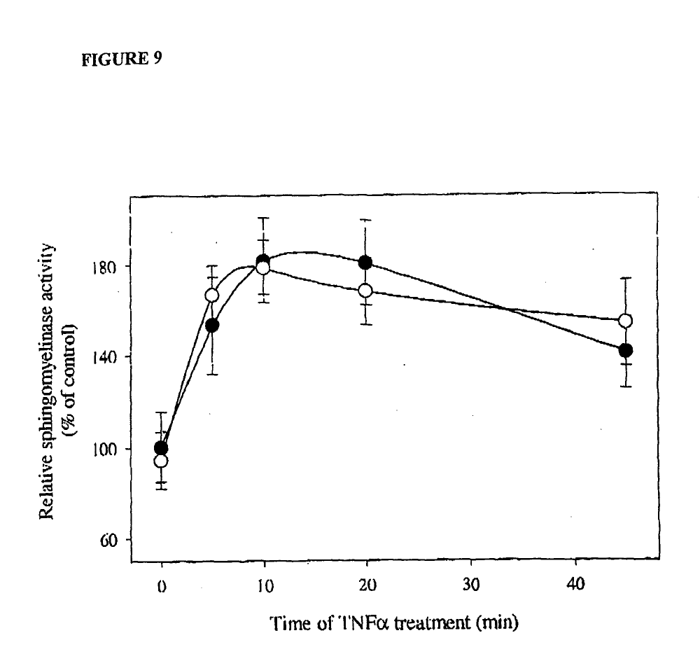

Furthermore, G82DSK inhibits sphingosine kinase stimulated by the oncogene Ras (FIG. 6) and may also suppress in vitro and in vivo markers of oncogenesis. The inhibitor is specific as it does not depress the activation of another enzyme protein kinase C (FIG. 7) or sphingomyelinase (FIG. 8). Furthermore, its function is stable as it inhibits TNF mediated activation at all time point (FIG. 4) and inhibits downstream effects of TNF such as activation of erk (FIG. 9).

Thus there has been generated an inhibitor of sphingosine kinase that is quite different from the chemical inhibitor hitherto used widely, N′N-dimethylsphingosine (DMS). DMS totally eliminates wild-type sphingosine kinase function whereas G82DSK only eliminates activation of wild-type sphingosine kinase. In a way, we have a prototype for a new kind of drug.

### EXAMPLE 3

Human sphingosine kinase (hSK) has been cloned and found, through sequence analysis to have, similarity in amino acids 16 to 153 to the putative catalytic domain of diacyglycerol kinases (DGKs) (Pitson et al., 2000a). Examples 1 and 2 disclose generation of a catalytically inactive mutant of hSK by site-directed mutagenesis of a single amino acid within this region (see also Pitson et al., 2000b). This mutation, Gly⁸² to Asp (referred to as G82D or hSK^{G82D}) was based on similar mutations of DGKs that also produced inactive mutants (Masai et al., 1993; Topham and Prescott, 1999; Topham et al., 1998). The mechanism whereby the point mutation ablated activity in DGKs and hSK has been proposed to be through altering the ATP binding site of the enzymes. This is based on the (loose) similarity in the amino acid sequence motifs for ATP binding sites in protein kinases (Hanks et al., 1988; Benner and Gerlo, 1992) and those found in DGKs and hSK. However, until now, no film data has been generated to support the involvement of Gly⁸² in ATP binding. This Example describes the generation of another hSK mutant through mutagenesis of Gly⁸² to Ala (G82A or hSK^{G82A}).

### Materials and Methods

Materials—D-erythro—Sphingosine was purchased from Biomol Research Laboratories Inc. (Plymouth Meeting, Pa.), ATP from Sigma, and [γ³²P]ATP from Geneworks (Adelaide, South Australia).

Cell Culture and Transfection—Human embryonic kidney cells (HEK293T, ATCC CRL-1573) cells were cultured on Dulbecco's modified Eagle's medium (DMEM; CSL Biosciences, Parkville, Australia) containing 10% fetal calf serum, 2 mM glutamine, 0.2% (w/v) sodium bicarbonate, penicillin (1.2 mg/ml), and gentamycin (1.6 mg/ml). Transfections were performed using the calcium phosphate precipitation method (Graham & van der Eb, 1973). Cells were harvested and lysed by sonication (2 watts for 30 s at 4° C.) in lysis buffer containing 50 mM Tris/HCl (pH 7.4), 10% glycerol, 0.05% Triton X-100, 150 mM NaCl, 1 mM dithiothreitol, 2 mM Na₃VO₄, 10 mM NaF, and 1 mM EDTA. Protein concentrations in cell homogenates were determined with the Coomassie Brilliant Blue (Sigma) reagent using BSA as standard.

Enzyme Assays—Sphingosine kinase activity was determined using D-erythro-sphingosine and [γ³²P]ATP as substrates, as described previously (Pitson et al., 2000a). Kinetic parameters were calculated using a non-linear regression program.

Construction of SK^{G82A}—The SKI cDNA (as described in Pitson et al., 2000a) was cloned into pALTER (Promega Inc., Madison, Wis.) site directed mutagenesis vector. Single-stranded DNA was prepared and used as a template for oligonucleotide directed mutagenesis as detailed in the manufacturer's protocol. The mutagenic oligonucleotide (5′-GTCTGGAGATGCATTGATGCACG-3′) was designed to generate the SK^{G82A} mutant, substitution of the glycine at position 82 to alanine. The mutant was sequenced to verify incorporation of the desired modification and sub-cloned into pcDNA3 (Invitrogen Corp., San Diego Calif.) for expression in HBEK293T cells.

Western Blotting—SDS-PAGE was performed on cell lysates according to the method of Laemmli (1970) using 12% acrylamide gels. Proteins were blotted to nitrocellulose and the membranes blocked overnight at 4° C. in PBS containing 5% skim milk and 0.1% Triton X-100. Sphingosine kinase expression levels were analysed with the M2 anti-FLAG antibody (Sigma). ERK activation in response to agonists was followed in cells serum-starved for 4 h using anti-ERK1/2 (Zymed, San Francisco, Calif.) and anti-phospho-ERK1/2 (Promega, Madison, Wis.) antibodies. Immunocomplexes were detected after conjugation to either HRP anti-mouse (Pierce) or anti-rabbit)(Selinus/AMRAD, Melbourne, Australia) IgG using an enhanced chemiluminescence kit (Amersham).

### EXAMPLE 4

### Results

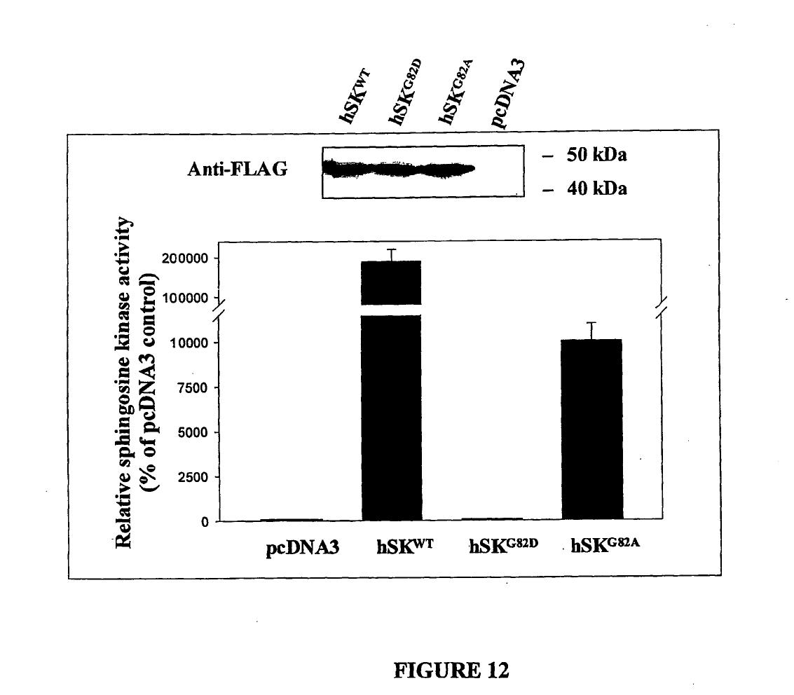

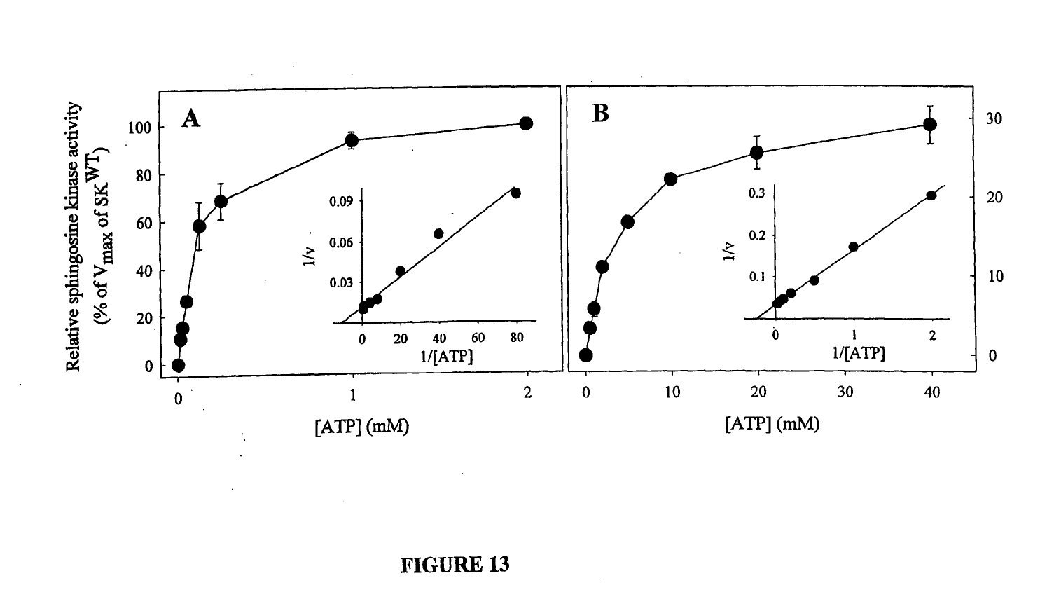

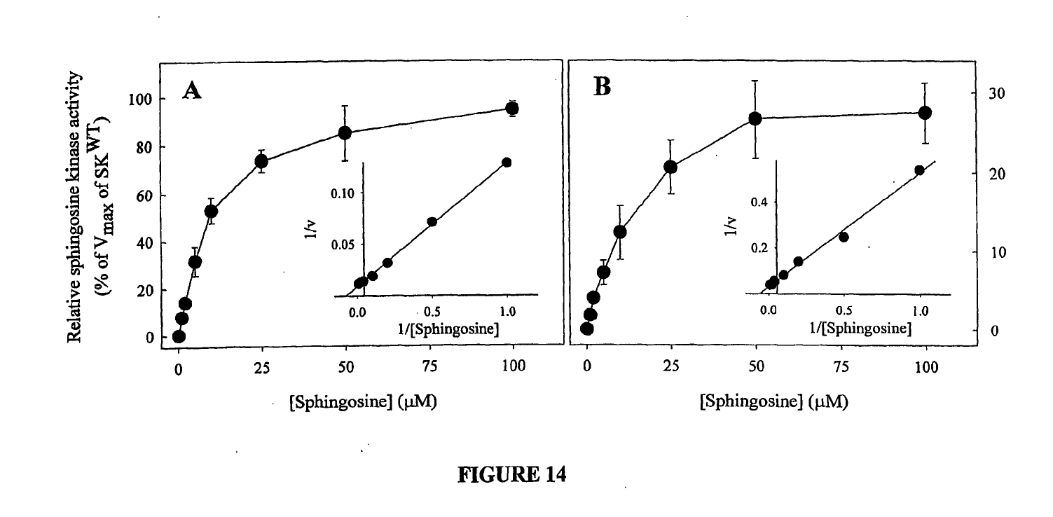

In contrast to the hSK^{G82D} mutant, hSK^{G82A} has catalytic activity, albeit much lower (ca 5%) than the wildtype hSK (FIG. 12). Analysis of the substrate kinetics of hSK^{G82A} has shown that this mutant has considerably lower affinity for ATP than the wildtype hSK (FIG. 13), while the affinity for sphingosine remains unaffected (FIG. 14). This kinetic data, summarised in Table 3, indicate that Gly⁸² is involved in ATP binding and suggests that this residue may be part of the ATP-binding site of hSK. This provides evidence that the original Gly⁸² to Asp mutation in hSK ablates catalytic activity of hSK by interruption of ATP binding.

### EXAMPLE 5

### Construction OF hSK Mutuants

Construction of hSK mutants—The SK1 cDNA (as described in Pitson et al., 2000a) was cloned into pALTER (Promega Inc., Madison, Wis.) site directed mutagenesis vector. Single-stranded DNA was prepared and used as a template for oligonucleotide directed mutagenesis as detailed in the manufacturer's protocol. The mutagenic oligonucleotides used to generate the hSK mutants are listed in Table 1. The mutants were sequenced to verify incorporation of the desired modification and sub-cloned into pcDNA3 (Invitrogen Corp., San Diego Calif.) for expression in HEK293T cells.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 3
Substrate kinetics of hSK^{WT} and hSK^{G82A}

Kₘ

| | ATP | Sphingosine | Vₘₐₓ |
|---|---|---|---|
| hSK^{WT} | 115 μM | 12.1 μM | 100 |
| hSK^{G82A} | 4.2 μM | 15.5 μM | 32 |

Kinetic values were obtained by non-linear regression analysis of data presented in FIGS. 12 and 13. Vₘₐₓ is expressed as a percentage of the maximum velocity calculated for SK^{WT} and standardised for the expression levels of the two recombinant proteins.

TABLE 4

Mutagenic oligonucleotides used for
site-directed mutagenesis of hSK
Mismatches with the hSKWT template
are indicated by lowercase letters.

| Name | Sequence | |
|---|---|---|
| G26A | GAACCCGCGGGGCGCCAAGGGCAA | (<400 > 13) |
| G26D | GCTGAACCCCCGGGGCGACAAGGGCAA | (<400> 14) |
| K27A | CGCGGCGGCGCCGGCAAGGCC | (<400 > 15) |
| K29A | GGCAAGGGCGCCGCCTTGCAG | (<400 > 16) |
| S79A | GTGGTCATGGCCGGCGACGGGCTG | (<400 > 17) |
| S79D | GTGGTCATGGATGGAGACGGCCTGATGCAC | (<400 > 18) |
| G80A | TCATGTCTGCAGACGGGCT | (<400 > 19) |
| G80D | TCATGTCTGACGACGGCCTGATGCAC | (<400 > 20) |
| G82A | GTCTGGAGATGCATTGATGCACG | (<400 > 21) |
| G82D | CTGGAGACGATCTGATGCAC | (<400 > 22) |
| K103A | GCCATCCAGGCCCCCCTGTGT | (<400 > 23) |
| K103R | GCCATCCAGCGGCCGCTGTGTAGC | (<400 > 24) |
| G111A | AGCCTCCCTGCAGCCTCTGGCAA | (<400 > 25) |
| G111D | TCCCAGCAGACTCTGGCAA | (<400 > 26) |
| G113D | CCCAGCAGGATCCGACAACGCGCT | (<400 > 27) |

### BIBLIOGRAPHY

Allessenko, A. V., (1998) Biochemistry (Mosc) 63:62-68.
Benner, S. A. and Gerloff, D. (1992) Adv. Enzyme Reg. 31:121-181.
Bock et al (1992), Nature 355:564-566.
Brenner and Lerner (1992), Proc. Natl. Acad. Sci. USA 89:5381-5383.
Christian, R. B. et al (1992), J. Mol. Biol. 227:711-718.
Culliver, O., Pirianov, G., Kleuser, B., Vanek, P. G., Coso, O. A., Gutkind, J. S, and Spiegel, S. (1996) Nature 381:800-803.
Devlin et al (1990), Science 249:404-406.
Ellington et al (1992), Nature 355:850-852.
Erb et al (1994), Proc. Natl. Acad. Sci. USA 91:11422-11426.
Fodor et al (1991), Science 251:767-773.
Fowlkes et al (1992), BioTechniques 13:422-427.
Gallop et al (1994), J. Medicinal Chemistry 37(9):1233-1251.
Graham, F. L. and van der Eb, A. J. (1973) Virology 54:536-539.
Hanks, S. K., Quinn, A. M. and Hunter, T. (1988) Science 241:42-52.
Houghten et al (1991), Nature 354:84-86.
Houghten et al (1992), Biotechniques 13:412.
Igarashi, Y. (1997) J. Biochem. 122:1080-1087.
Jayawickreme et al (1994), Proc. Natl. Acad Sci. USA 91:1614-1618.
Kay et al (1993), Gene 128:59-65.
Laemmli, U. K. (1970) Nature 227:680-685.
Lam et al (1991), Nature 354:82-84.
Lenstra (1992), J. Immunol. Meth 152:149-157.
Masai, I., Okazaki, A., Kosoya, T. and Hotta, Y. (1993) Proc. Natl. Acad. Sci. USA 90:11157-11161.
Mattheakis et al (1994), Proc. Natl. Acad. Sci. USA 91:9022-9026.
Medynski (1994), Bio/Technology 12:709-710.
Meyer zu Heringdorf, D., van Koppen, C. J. and Jakobs, K. H. (1997) FEBS Lett 410:34-38.
Ohlmeyer et al (1993), Proc. Natl. Acad. Sci. USA 90:10922-10926.
Oldenburg et al (1992), Proc. Natl. Acad. Sci USA 89:5393-5397.
Parmley and Smith (1989), Adv. Exp. Med. Biol. 251:215-218.
Pitson, S. M., D'Andrea, R. J., Vandeleur, L., Moretti, P. A. B., Xia, P., Gamble, J. R., Vadas, M. A. and Wattenberg, B. W. (2000a) Biochemical Journal 350:429-441.
Pitson, S. M., Moretti, P. A. B., Zebol, J. R, Xia, P., Gamble, J. R, Vadas, M. A., D'Andrea, R. J. and Wattenberg, B. W. (2000b) J. Biol. Chem. in press.
Rebar and Pabo (1993), Science 263:671-673.
Salmon et al (1993), Proc. Natl. Acad. Sci. USA 90:11708-11712.
Scott and Smith (1990), Science 249:386-390.
Scott and Smith (1990), Science 249:386-390.
Spiegel, S., Culliver, O., Edsall, L., Kohama, T., Menzeleev, R., Olivera, A., Thomas, D., Tu, Z., Van Brocklyn, J. and Wang, F. (1998) Biochemistry (Mosc) 63:69-73.
Staudt et al (1988), Science 241:577-580.
Topham, M. K. and Prescott, S. M. (1999) J. Biol. Chem. 274:11447-11450.
Topham, M. K., Bunting, M. Zimmerman, G. A., McIntyre, T. M., Blackshear, P. J., and Prescott, S. M. (1998) Nature 394:697-700.
Tuerk et al (1992), Proc. Natl. Acad. Sci. USA 89:6988-6992.
Weigmann, K., Schutze, S., Machleidt, T., Witte, D. and Kronke, M. (1994) Cell 78:1005-1015.
Xia, P., Aiello, L. P., Ishii, H., Jiang, Z. Y., Park, D. J., Robinson, G. S., Takagi, H., Newsome, W. P., Jirousek, M. R. and King, g. L. (1996) Journal of clinical Investigation 98:2018-2026.
Xia, P., Gamble, J. R., Rye, K.-A., Wang, L., Hii, C. S. T., Cockerill, P., Khew-Goodall, Y., Bert, A. G., Barter, P. J., and Vadas, M. A. (1998) Proc. Natl. Acad, Sci. USA 95:14196-14201.
Yu et al (1994), Cell 76:933-945.

1. A sphingosine kinase variant comprising a mutation in a region defined by amino acids 16-153 or a functionally equivalent region wherein said variant exhibits ablated or reduced catalytic activity relative to a wild-type sphingosine kinase or a derivative, homologue, analogue, chemical equivalent or mimetic of said sphingosine kinase variant.

2. The sphingosine kinase variant according to claim 1 wherein said sphingosine kinase is a human sphingosine kinase.

3. The sphingosine kinase variant according to claim 1 wherein said mutation comprises a single or multiple amino acid substitution, addition, deletion, or combination thereof.

4. The sphingosine kinase variant according to claim 3 wherein said region is defined by amino acids 70-90.

5. The sphingosine kinase variant according to claim 4 wherein said region is defined by amino acids 79-84.

6. The sphingosine kinase variant according to claim 5 wherein said mutation is an amino acid substitution of a glycine amino acid at position 82 to an aspartic acid.

7. The sphingosine kinase variant according to claim 1 wherein said variant exhibits ablated catalytic activity.

8. The sphingosine kinase variant according to claim 7 wherein said variant comprises one or more of the amino acid substitutions selected from the group consisting of:
(i) G82D;
(ii) .G82A;
(iii) G26D;
(iv) S79D;
(v) G80D;
(vi) K103A;
(vii) G111D;
(viii) G113D;
(ix) G26A;
(x) K27A;
(xi) K29A;
(xii) S79A;
(xiii) G80A;
(xiv) K103R; and
(xv) G111A.

9. A sphingosine kinase variant comprising a mutation in an ATP binding site region or a functionally equivalent region wherein said variant exhibits ablated or reduced catalytic activity relative to a wild-type sphingosine kinase or a derivative, homologue, analogue, chemical equivalent or mimetic of said sphingosine kinase variant.

10. The sphingosine kinase variant according to claim 9 wherein said sphingosine kinase is a human sphingosine kinase.

11. The sphingosine kinase variant according to claim 9 wherein said mutation comprises a single or multiple amino acid substitution, addition, deletion, or combination thereof.

12. The sphingosine kinase variant according to claim 11 wherein said region is defined by amino acids 70-90.

13. The sphingosine kinase variant according to claim 12 wherein said region is defined by amino acids 79-84.

14. The sphingosine kinase variant according to claim 9 wherein said mutation is an amino acid substitution of a glycine amino acid at position 82 to an aspartic acid.

15. The sphingosine kinase variant according to claim 9 wherein said variant exhibits ablated catalytic activity.

16. The sphingosine kinase variant according to claim 15 wherein said variant comprises one or more amino acid substitutions selected from the group consisting of:
(i) G82D;
(ii) G82A;
(iii) G26D;
(iv) S79D;
(v) G80D;
(vi) K103A;
(vii) G111D;
(viii) G113D;
(ix) G26A;
(x) K27A;
(xi) K29A;
(xii) S79A;
(xiii) G80A;
(xiv) K103R; and
(xv) G111A.

17. An isolated nucleic acid molecule selected from the group consisting of:
(i) a nucleotide sequence encoding or complementary to a sequence encoding a sphingosine kinase variant or derivative, homologue, analogue, chemical equivalent or mimetic of said variant which variant comprises a mutation in a region defined by amino acid 16-153 or a functionally equivalent region wherein said variant exhibits ablated or reduced catalytic activity relative to a wild-type sphingosine kinase;
(ii) a nucleotide sequence encoding or complementary to a sequence encoding a human sphingosine kinase variant or derivative, homologue, analogue, chemical equivalent or mimetic of said variant which variant comprises a mutation in a region defined by amino acid 16-153 or a functionally equivalent region wherein said variant exhibits ablated or reduced catalytic activity relative to a wild-type human sphingosine kinase;
(iii) a nucleotide sequence encoding or complementary to a sequence encoding a human sphingosine kinase variant or derivative, homologue, analogue, chemical equivalent or mimetic of said variant, which variant comprises an amino acid sequence with a single or multiple amino acid substitution, addition or deletion in a region defined by amino acid 16-153 or a functionally equivalent region wherein said variant exhibits ablated or reduced catalytic activity relative to a wild-type sphingosine kinase;
(iv) a nucleotide sequence encoding or complementary to a sequence encoding a human sphingosine kinase variant or derivative, homologue, analogue, chemical equivalent or mimetic of said variant, which variant comprises an amino acid sequence with a single or multiple amino acid substitution, addition or deletion in a region defined by amino acid 70-90 or a functionally equivalent region wherein said variant exhibits ablated or reduced catalytic activity relative to a wild-type sphingosine kinase;
(v) a nucleotide sequence encoding or complementary to a sequence encoding a human sphingosine kinase variant or derivative, homologue, analogue, chemical equivalent or mimetic of said variant, which variant comprises an amino acid sequence with a single or multiple amino acid substitution, addition or deletion in a region defined by amino acid 79-84 or a functionally equivalent region wherein said variant exhibits ablated or reduced catalytic activity relative to a wild-type sphingosine kinase;
(vi) a nucleotide sequence encoding or complementary to a sequence encoding a sphingosine kinase variant or a derivative, homologue, analogue, chemical equivalent or mimetic of said variant comprising one or more of the amino acid substitutions selected from the group consisting of:
(a) G82D;
(b) G82A;
(c) G26D;
(d) S79D;
(e) G80D;
(f) K103A;
(g) G111D;
(h) G113D;
(i) G26A;
(h) G113D;
(i) G26A;
(j) K29A;
(k) S79A;
(l) G80A;
(m) K103R; and
(n) G111A; and
(vii) a nucleotide sequence encoding or complementary to a sequence encoding a sphingosine kinase variant or derivative, homologue, analogue, chemical equivalent or mimetic of said variant which variant comprises a mutation in an ATP binding site region or a functionally equivalent region wherein said variant exhibits ablated or reduced catalytic activity relative to a wild-type sphingosine kinase.

18. A method for detecting an agent capable of modulating the interaction of FOSK with a sphingosine kinase or a functional equivalent or derivative thereof said method comprising contacting a cell or extract thereof containing said sphingosine kinase and FOSK or said functional equivalent or derivative with a putative agent and detecting an altered expression phenotype associated with said interaction.

19. A method for detecting an agent capable of binding or otherwise associating with a sphingosine kinase region defined by amino acid 16-153 or a functional equivalent or derivative thereof said method comprising contacting a cell containing said amino acid region or the functional equivalent or derivative thereof with a putative agent and detecting an altered expression phenotype associated with modulation of a function of said sphingosine kinase or a functional equivalent or derivative thereof.

20. The method according to claim 19 wherein said amino acid region is defined by amino acid 70-90.

21. The method according to claim 20 wherein said amino acid region is defined by amino acid 79-84.

22. A method for analyzing, designing or modifying an agent capable of interacting with a sphingosine kinase region defined by amino acid 16-153 or a derivative thereof and modulating at least one functional activity associated with said sphingosine kinase said method comprising contacting said sphingosine kinase or derivative thereof with a putative agent and assessing the degree of interactive complementarity of said agent with said binding site.

23. The method according to claim 22 wherein said amino acid region is defined by amino acid 70-90.

24. The method according to claim 23 wherein said amino acid region is defined by amino acid 79-84.

25. The putative agent identified in accordance with the method of anyone of claims 18-24.

26. A method of modulating cellular functional activity in a mammal said method comprising administering to said mammal an effective amount of the sphingosine kinase variant according to anyone of claims 1-17 for a time and under conditions sufficient to inhibit, reduce or otherwise down-regulate at least one functional activity of a wild-type sphingosine kinase.

27. The method according to claim 26 wherein said activity is down-regulation of wild-type sphingosine kinase baseline activity or prevention of wild-type sphingosine kinase activation.

28. A method for treatment or prophylaxis of a condition in a mammal, which condition is characterized by aberrant, unwanted or otherwise inappropriate cellular activity, said method comprising administering to said mammal an effective amount of the sphingosine kinase variant according to anyone of claims 1-17 or agent according to claim for a time and under conditions sufficient to inhibit, reduce or otherwise down-regulate at least one functional activity of said wild-type sphingosine kinase wherein said down-regulation results in modulation of a cellular functional activity.

29. The method according to claim 28 wherein said activity is down-regulation of wild-type sphingosine kinase baseline activity or prevention of wild-type sphingosine kinase activation.

30. A method for manufacture of a medicament for the modulation of a cellular functional activity comprising the sphingosine kinase variant according to anyone of claims 1-17.

31. A method for modulating a cellular functional activity comprising the sphingosine kinase variant according to anyone of claims 1-17.

32. A pharmaceutical composition comprising the sphingosine kinase variant according to anyone of claims 1-17 together with one or more pharmaceutically acceptable carriers or diluents.

33. A method of modulating cellular functional activity in a mammal said method comprising administering to said mammal an effective amount of the putative agent according to claim 25 for a time and under conditions sufficient to inhibit, reduce or otherwise down-regulate at least one functional activity of a wild-type sphingosine kinase.

34. The method according to claim 33 wherein said activity is down-regulation of a wild-type sphingosine kinase baseline activity or prevention of a wild-type sphingosine kinase activation.

35. A method for treatment or prophylaxis of a condition in a mammal, which condition is characterized by aberrant, unwanted or otherwise inappropriate cellular activity, said method comprising administering to said mammal an effective amount of the putative agent according to claim 25 for a time and under conditions sufficient to inhibit, reduce or otherwise down-regulate at least one functional activity of a wild-type sphingosine kinase wherein said down-regulation results in modulation of a cellular functional activity.

36. The method according to claim 35 wherein said activity is down-regulation of a wild-type sphingosine kinase baseline activity or prevention of a wild-type sphingosine kinase activation.

37. A method for manufacture of a medicament for the modulation of cellular functional activity comprising the agent according to claim 25.

38. A method for modulating cellular functional activity comprising the agent according to claim 25.

39. A pharmaceutical composition comprising the agent according to claim 25 together with one or more pharmaceutically acceptable carriers or diluents.
